(19) United States
(12) Patent Application Publication
(10) Pub. No.: US 2017/0131287 A1
(43) Pub. Date: May 11, 2017

(54) SACCHARIDE RESPONSIVE OPTICAL NANOSENSORS

(71) Applicant: Massachusetts Institute of Technology, Cambridge, MA (US)

(72) Inventors: Thomas P. McNicholas, Cambridge, MA (US); Jiyoung Ahn, Cambridge, MA (US); Michael S. Strano, Lexington, MA (US)

(73) Assignee: Massachusetts Institute of Technology, Cambridge, MA (US)

(21) Appl. No.: 15/318,337

(22) PCT Filed: Jun. 5, 2015
(86) PCT No.: PCT/US2015/034445

§ 371 Date: Dec. 12, 2016

Related U.S. Application Data
(60) Provisional application No. 62/011,885, filed on Jun. 13, 2014.

(51) Int. Cl. G01N 33/66; G01N 33/58

(52) U.S. Cl. CPC G01N 33/66; G01N 33/582

(57) ABSTRACT

A composition for sensing an analyte can include a photoluminescent nanostructure (e.g. a carbon nanotube) complexed to a sensing polymer, where the sensing polymer includes a phenylboronic acid based polymer non-covalently bound to the photoluminescent nanostructure where the composition is capable of selectively binding the analyte, and the composition undergoes a substantial conformational change when binding the analyte. Separately, a composition for sensing an analyte can include a complex, where the complex include a photoluminescent nanostructure in an aqueous surfactant dispersion and a phenylboronic acid capable of selectively reacting with an analyte. The compositions can be used in devices and methods for sensing an analyte.

### CLAIM OF PRIORITY

This application claims the benefit of prior U.S. Provisional Application No. 62/011,885 filed on Jun. 13, 2014, which is incorporated by reference in its entirety.

### TECHNICAL FIELD

The present invention generally relates to sensors based on photoluminescent nanostructures.

### BACKGROUND

In vivo sensors are of particular interest in the biomedical field, where continuous and/or real time patient data can be desirable; in particular, sensors that can detect and measure the levels of biological compounds (e.g., metabolites). Such sensors can involve a sensor material that interacts with an analyte, where the interaction results in changes in how the sensor material interacts with light, e.g., changes in the absorption or luminescence properties of the sensor material. However, many proposed methods are expensive, require high resolution, and involve the use of bulky equipment.

Diabetes affects nearly 17.9 million people in the United States alone, with 1.6 million new cases being diagnosed each year. Diabetes was the seventh leading cause of death in the United States as of 2006, and is still rising. Current treatments involve monitoring of glucose levels in a patient's body. This monitoring allows the patient to appropriately treat glucose levels which are outside of the safe range, and thus avoid complications which could otherwise result.

The basic glucose monitoring device in use today, a finger-stick glucose monitor, has certain disadvantages. These include the pain associated with the finger stick, and the discontinuous nature of the information provided. With such devices, a patient must rely on a few single-point measurements taken throughout the day to monitor his or her blood glucose levels. Accordingly, there remains a need for a real-time, continuous blood glucose monitor.

### SUMMARY

Sensors based on photoluminescent nanostructures, and methods of making and using them, are described. Photoluminescent nanostructures (e.g., single-walled carbon nanotubes, or SWNTs) can be combined with an analyte-binding group in such a way that the photoluminescence is altered when the analyte interacts with the analyte binding group. For example, when the analyte in question is glucose, the analyte binding group can be a glucose binding protein or a boronic acid. The photoluminescent nanostructures can be packaged in a biocompatible matrix suitable for use in vivo to produce a real-time, continuous and long-term glucose monitor.

In one aspect, a composition for sensing an analyte can include a photoluminescent nanostructure complexed to a sensing polymer, wherein the sensing polymer can be a copolymer including monomer units having a boronic acid moiety and non-covalently bound to the photoluminescent nanostructure, where the composition is capable of selectively binding the analyte, and the composition undergoes a substantial conformational change when binding the analyte. The sensing polymer can be a poly acrylic polymer and the poly acrylic polymer can include a boronic acid moiety. The photoluminescent nanostructure can be a carbon nanotube, such as single wall nanotube (SWNT). The boronic acid moiety can be a phenylboronic acid. The analyte can be a saccharide, such as glucose.

In another aspect, a method of synthesizing a composition for sensing an analyte can include selecting a concentration of initiator and a boronic acid derivative, conducting polymerization of a monomer and the boronic acid derivative, where the resulting polymer has a selectivity to an analyte, and mixing with a photoluminescent nanostructure. The boronic acid derivative can be included in a poly acrylic polymer. The photoluminescent nanostructure can be a carbon nanotube, such as single wall nanotube (SWNT). The boronic acid moiety can be a phenylboronic acid. The analyte can be a saccharide, such as glucose. The monomer can be 4-vinylphenylboronic acid, 3-vinylphenylboronic acid, 2-vinylphenylboronic acid, maleic anhydride, or styrene.

In another aspect, a method for sensing an analyte can include providing a composition, wherein the composition includes, a photoluminescent nanostructure complexed to a sensing polymer, where the sensing polymer is a copolymer including a monomer units having a boronic acid moiety and non-covalently bound to the photoluminescent nanostructure, where the composition is capable of selectively binding the analyte, and the composition undergoes a substantial conformational change when binding the analyte, and contacting the composition with a sample suspected of containing the analyte. The sensing polymer can be a poly acrylic polymer and the poly acrylic polymer can include a boronic acid moiety. The photoluminescent nanostructure can be a carbon nanotube, such as single wall nanotube (SWNT). The boronic acid moiety can be a phenylboronic acid. The analyte can be a saccharide, such as glucose.

A composition for sensing an analyte can include a complex, where the complex includes a photoluminescent nanostructure in an aqueous dispersion and a boronic acid capable of selectively reacting with an analyte. The boronic acid can be included in a poly acrylic polymer. The photoluminescent nanostructure can be a carbon nanotube, such as single wall nanotube (SWNT). The boronic acid moiety can be a phenylboronic acid. The analyte can be a saccharide, such as glucose.

A device for sensing an analyte can include a hydrogel particle encapsulating a composition, wherein the composition includes a complex, wherein the complex includes a photoluminescent nanostructure in an aqueous dispersion and a boronic acid capable of selectively reacting with an analyte. The boronic acid can be included in a poly acrylic polymer. The photoluminescent nanostructure can be a carbon nanotube, such as single wall nanotube (SWNT). The boronic acid moiety can be a phenylboronic acid. The analyte can be a saccharide, such as glucose.

In another aspect, a method for sensing an analyte can include providing a composition, wherein the composition includes a complex, where the complex includes a photoluminescent nanostructure in an aqueous dispersion and a boronic acid containing polymer capable of selectively reacting with an analyte, and contacting the composition with a sample suspected of containing the analyte. The boronic acid containing polymer can be included in a poly acrylic polymer. The photoluminescent nanostructure can be a carbon nanotube, such as single wall nanotube (SWNT). The boronic acid moiety can be a phenylboronic acid. The analyte can be a saccharide, such as glucose.

A composition of a polymer can include a poly acrylic acid backbone and a boronic acid moiety. The boronic acid moiety can be a phenylboronic acid.

Other aspects, embodiments, and features will become apparent from the following description, the drawings, and the claims.

### BRIEF DESCRIPTION OF THE DRAWINGS

FIG. 1 is a schematic of water soluble phenylboronic acid based polymer synthesis and subsequent association with SWNT.

FIG. 2 is a photograph of polymer SWNT suspensions and both photoabsorption and nIR fluorescent spectra observed from each polymer-SWNT suspensions.

FIG. 3A is a schematic depiction of a 4-vinyl phenylboronic acid polymer derivative-SWNT complex. FIGS. 3B-3C is a graph depicting fluorescent quenching response after the addition of glucose. FIG. 3D is a photoabsorption spectrum of the SWNT complex before and after glucose addition, indicating no change in the stability of the SWNT suspension.

FIG. 4 is a diagram showing photoabsorption (A) induces an electronic excitement of the SWNT.

FIGS. 5A-5D are graphs showing the saccharide binding profiles of all polymers-SWNT are distinct both from one another and from the free polymers.

FIG. 6 is a calibration curve demonstrating the sensitivity of 3-PBA-hMA-1-SWNT to D-(+)-glucose.

FIG. 7 are graphs depicting NMR analysis confirming polymer formation in each case yielding approximately a 1:1 ratio of monomers.

FIG. 8 is graphs showing the characterization of films made from hydrolyzed polymer solutions of each polymer system using Fourier Transform Infrared Spectroscopy.

FIG. 9 is a photograph and graphs showing that simple stirring in either nanopure water or PBS buffer hydrolyzes the formed polymer.

FIG. 10 are graphs showing that ARS binding studies illustrate the conserved ability of the PBA monomer to form diol bonds.

FIG. 11 are graphs showing that Fluorescent excitation/emission mapping demonstrates the successful SWNT suspension formation.

FIG. 12 are graphs showing that plotting E₁₁ v 1/d⁴ allows for the assignment of relative SWNT surface coverage assuming 100% surface coverage by NMP.

FIG. 13 are graphs showing that saccharide screening done at pH=1 demonstrates that significantly changing the pH alters the binding profile of each polymer-SWNT system.

FIG. 14 are graphs depicting calibration curves for saccharides.

FIG. 15A shows structures of three sugar alcohols tested. FIG. 15B shows the relation between the response to sugar alcohol and the location of the boronic acid.

### DETAILED DESCRIPTION

Sensors based on photoluminescent nanostructures, and methods of making and using them, are described. Photoluminescent nanostructures (e.g., single-walled carbon nanotubes, or SWNTs) can be combined with an analyte-binding group in such a way that the photoluminescence is altered when the analyte interacts with the analyte binding group. For example, when the analyte in question is glucose, the analyte binding group can be a glucose binding protein or a boronic acid. The photoluminescent nanostructures can be packaged in a biocompatible matrix suitable for use in vivo to produce a real-time, continuous and long-term glucose monitor.

In general, an analyte sensing composition can include photoluminescent nanostructure in a complex (e.g., a non-covalent complex) with a polymer, such as a sensing polymer. The photoluminescent nanostructure can be a carbon nanotube. A sensing polymer can include, for example, an organic polymer (including but not limited to poly(alkylene glycols) (e.g., poly(ethylene glycol)), poly(vinyl alcohol), carboxylated poly(vinyl alcohol), poly(vinyl chloride), polysorbitan esters (e.g., polyoxyethylene sorbitan fatty acid esters), and copolymers of these, whether with each other or with other polymers), a protein, a polypeptide, a polysaccharide or a poly acrylic acid polymer (e.g. a polymer displaying a phenyl boronic acid).

The poly acrylic acid polymer can have modifications such as possessing a phenyl ring off the backbone. The poly acrylic acid polymer can display one or two carboxylic acids per monomer unit and can display 0, ½ or 1 boronic acid per unit cell. The phenyl boronic acid can be ortho-, meta- or para- on the ring. The structural differences of the phenyl boronic acid can make a difference in analytes that can be recognized. The length of the polymer can be important for the analyte recognition. [Please describe in more detail if possible] The polymers with longer lengths can be preferred for sugar alcohol recognition and detection. The analyte can be a structure that moderately recognizes glucose, a structure that recognizes sucrose but not glucose or fructose, or a structure that recognizes sorbitol and ducitol over mannitol.

In the sensing composition, the sensing polymer can complexed with the carbon nanotube to provide individually dispersed carbon nanotubes with no electronic interaction or minimal electronic interaction with other carbon nanotubes in the composition. The sensing polymer can selectively interact with an analyte. The term “selective” indicates an interaction that can be used to distinguish the analyte in practice from other chemical species, even species which may be structurally related or similar to the analyte, in the system in which the sensor and sensing composition is to be employed.

The interaction can be, for example, a reversible or irreversible non-covalent binding interaction; a reversible or irreversible covalent binding interaction (i.e., a reaction wherein a covalent bond between the sensing polymer and the analyte is formed); or catalysis (e.g., where the sensing polymer is an enzyme and the analyte is a substrate for the enzyme).

The term “selective binding” is thus used to refer to an interaction, typically a reversible non-covalent binding interaction, between a sensing polymer and an analyte, which is substantially stronger than the interaction between the sensing polymer and species that are related in chemical structure to the analyte. The strength of a selective binding interaction may be determined with reference to, for example, an equilibrium binding constant for a given set of conditions.

Enzymes, antibodies (and antibody fragments) and receptors, among other proteins, can exhibit specific binding which may in some cases be selective. Other polymers, such as polysaccharides may function as ligands (e.g., for binding to a protein) or as a member of a binding pair. Selective binding can provide the selectivity needed to detect a selected analyte (or relatively small group of related analytes) in a complex mixture, e.g., in a biological fluid or tissue. For example, selective binding of a substrate to an enzyme can provide the desired level of selectivity needed to detect a selected analyte (which is the enzyme substrate). Sensing polymers can be chosen to provide selective interactions with one or more analytes. Preferably a particular sensing polymer can have a selective interaction with just one analyte; in other words, the selectivity is such that the sensing polymer can distinguish between the analyte and virtually all other chemical species.

The term “analyte” refers to any chemical species, suspected of being present in a sample, which the presence or absence of in the sample is to be determined, or the quantity or concentration of in the sample is to be determined. Analytes can include small molecules, such as sugars, steroids, antigens, metabolites, drugs, and toxins; and polymeric species such as proteins (e.g., enzymes, antibodies, antigens). In specific embodiments, analytes are one member of a binding partner pair. In some embodiments, analytes are monosaccharides, e.g., glucose. The compositions, methods, and systems described can be particularly well suited to the detection and/or quantitation of analytes in solutions, such as biological fluids. The compositions, methods, and systems described can also be particularly well suited to the detection and/or quantitation of analytes in biological tissues, including tissues in vivo.

The sensing polymer can be formed by derivatization of a polymer with one or more chemically selective species which provide for selective or specific interaction with one or more analytes. Polymers that may be derivatized to form sensing polymers include, but are not limited to, poly(alkylene glycols) such as poly(ethylene glycol), poly(vinyl alcohol), poly(vinyl chloride), polysorbitan esters (e.g., polyoxyethylene sorbitan fatty acid esters), and copolymers of these, whether with each other or with other polymers. Each sensing polymer may be derivatized to carry one or more chemically selective species or moieties which are each selective for the same analyte. A sensing polymer may be derivatized to carry one or more chemically selective species or moieties which are each selective for a different analyte. Thus a single composition may be responsive to a single analyte, or to more than one different analytes. In specific embodiments, a sensing polymer contains covalently bound, chemically selective species or moieties selective for a single analyte of interest. The use of polymers which carry one such selective chemical species or moiety may be beneficial to prevent aggregation of the complexes of the photoluminescent nanostructure and the sensing polymer. Such aggregation can be detrimental in analyte sensing applications. The chemically selective species or moiety may be directly bonded to the polymer or indirectly bonded through a linker group.

The sensing polymer can be a sensing protein. The sensing protein may be a naturally-occurring protein or recombinant protein that exhibits a selective interaction with an analyte. The sensing protein can interact directly with an analyte (e.g., by binding or reaction) or can interact indirectly with the analyte by interaction (e.g., by binding or reaction) with another chemical species which in turn interacts with the analyte. The sensing protein may be formed by chemical derivatization of a protein that does not exhibit any selective interaction with an analyte. For example, the sensing protein may be formed from a protein that is derivatized covalently to carry one or more chemically selective species (or moieties) which individually or collectively provide for selective interaction with one or more analytes. Proteins may be derivatized at one or more termini or at one or more amino acid side changes (e.g., those of lysine, glutamine, arginine, serine, aspartate, glutamate, etc.) to provide for chemical selectivity.

For some proteins, binding of the analyte causes a substantial conformational change in the protein. A substantial conformational change is one that causes a relatively large movement of one or more substructures of the protein. For example, a substantial conformational change can involve a relative movement of domains of the protein, or a relative movement of subunits of a multimeric protein. In some cases, the protein can be considered to have distinct conformations, depending on whether or not the analyte is bound. For example, some proteins can be described as being in an “open” or “closed” state depending on whether or not the analyte is bound; “open” and “closed” can describe the relative size of a cleft between two domains (i.e., the cleft is larger or more “open” in one state and smaller or more “closed” in another state).

Without intending to be bound by a particular theory, in the context of a sensor, the substantial conformational change can affect the photoluminescence properties (e.g., intensity or peak wavelength) of a photoluminescent nanostructure. The substantial conformational change can provide a mechanical force or actuation on the photoluminescent nanostructure; in other words, the substantial conformational change alters how the sensing protein interacts with or impinges on the photoluminescent nanostructure, which in turn affects the photoluminescence properties.

A sensing polymer can provide for selective interaction with an analyte. The sensing polymer may be naturally occurring, for example isolated from nature, chemically derivatized, chemically modified, or chemically synthesized. The sensing polymer can interact directly with an analyte (e.g., by binding or reaction) or can interact indirectly with the analyte by interaction (e.g., by binding or reaction) with another chemical species which in turn interacts with the analyte. The specific structure of the polysaccharide or the presence of a specific monosaccharide may facilitate a selective interaction with an analyte. The sensing polymer may be formed by chemical derivatization or modification of a polysaccharide that does not exhibit any selective interaction with an analyte. For example, the sensing polymer may be formed from a polysaccharide that is derivatized covalently to carry one or more chemically selective species (or moieties) which individually or collectively provide for selective interaction with one or more analytes. Polysaccharides may be derivatized at any available location of the polymer that is reactive to provide for chemical selectivity. Polysaccharides that are useful, for example, as sensing polymers include those polysaccharides which bind to a binding partner, for example a protein, that also binds to a monosaccharide analyte. Polysaccharides include those having 10 or more monosaccharide units, 20 or more monosaccharide units, 10 or more disaccharide units, or 20 or more disaccharide units.

As used herein, the term “nanostructure” refers to articles having at least one cross-sectional dimension of less than about 1 μm, less than about 500 nm, less than about 250 nm, less than about 100 nm, less than about 75 nm, less than about 50 nm, less than about 25 nm, less than about 10 nm, or, in some cases, less than about 1 nm. Examples of nanostructures include nanotubes (e.g., carbon nanotubes), nanowires (e.g., carbon nanowires), graphene, and quantum dots, among others. In some embodiments, the nanostructures include a fused network of atomic rings.

A “photoluminescent nanostructure,” as used herein, refers to a class of nanostructures that are capable of exhibiting photoluminescence. Examples of photoluminescent nanostructures include, but are not limited to, carbon nanotubes (e.g., single-walled and double-walled carbon nanotubes), semiconductor quantum dots, semiconductor nanowires, and graphene, among others. In some embodiments, photoluminescent nanostructures exhibit fluorescence. In some instances, photoluminescent nanostructures exhibit phosphorescence.

Carbon nanotubes are carbon nanostructures in the form of tubes, generally ranging in diameter from about 0.5-200 nm, (more typically for single-walled carbon nanotubes from about 0.5-5 nm) The aspect ratio of nanotube length to nanotube diameter is greater than 5, ranges from 10-2000 and more typically 10-100. Carbon nanotubes may be single-walled nanotubes (a single tube) or multi-walled comprising with one or more smaller diameter tubes within larger diameter tubes. Carbon nanotubes are available from various sources, including commercial sources, or synthesis employing, among others, arc discharge, laser vaporization, the high pressure carbon monoxide processes.

The following references provide exemplary methods for synthesis of carbon nanotubes: U.S. Pat. No. 6,183,714; WO/2000/026138; WO/2000/017102; A. Thess et al. Science (1996) 273:483; C. Journet et al. Nature (1997) 388, 756; P. Nikolaev et al. Chem. Phys. Lett. (1999) 313:91; J. Kong et al. Chem. Phys. Lett. (1998) 292: 567; J. Kong et al. Nature (1998) 395:878; A. Cassell et al. J. Phys. Chem. (1999) 103:6484; H. Dai et al. J. Phys. Chem. (1999) 103:11246; Bronikowski, M. J., et al., Gas-phase production of carbon single-walled nanotubes from carbon monoxide via the HiPco process: a parametric study. J. Vac. Sci. Tech. A, 2001. 19(4): p. 1800-1804; Y. Li et al. (2001) Chem. Mater. 13:1008; N. Franklin and H. Dai (2000) Adv. Mater. (2000) 12:890; A. Cassell et al. J. Am. Chem. Soc. (1999) 121:7975; and International Patent Applications WO 00/26138, WO 03/084869, and WO 02/16257; each of which is incorporated by reference in its entirety. Carbon nanotubes produced in such methods are typically poly-disperse samples containing metallic and semi-conducting types, with characteristic distributions of diameters.

A method for separating single-walled carbon nanotubes by diameter and conformation based on electronic and optical properties has been reported (WO 03/084869, which is incorporated by reference in its entirety. The method can be employed to prepare carbon nanotube preparations having enhanced amounts of certain single walled carbon nanotube types. Narrow (n, m)-distributions of single-walled carbon nanotubes are reported using a silica-supported Co—Mo catalyst. M. Zheng et al. Science (2003) 302 (November) 1545 (which is incorporated by reference in its entirety) report nanotube separation by anion exchange chromatography of carbon nanotubes wrapped with single-stranded DNA. Early fractions are reported to be enriched in smaller diameter and metallic nanotubes, while later fractions are enriched in larger diameter and semi-conducting nanotubes.

Carbon nanotube compositions generally useful in sensors can exhibit optical properties which are sensitive to the environment of the nanotube, i.e., optical properties which can be modulated by changes in the environment of the nanotube. More specifically, carbon nanotube compositions useful in sensors can be SWNTs, particularly semiconducting SWNTs, which can exhibit luminescence, and more specifically which exhibit photo-induced band gap fluorescence. Carbon nanotube compositions which exhibit luminescence include SWNTs which when electronically isolated from other carbon nanotubes exhibit luminescence, including fluorescence and particularly those which exhibit fluorescence in the near-IR. Carbon nanotube compositions can include individually dispersed semiconducting SWNTs exhibiting luminescence, particularly photo-induced band gap fluorescence. Carbon nanotube compositions may also include MWNT and other carbon nanomaterials as well as amorphous carbon. Preferably carbon nanotube compositions can include a substantial amount of semiconducting SWNTs, e.g., 25% or more, or 50% or more by weight of such SWNTs. In general, carbon nanotube compositions will contain a mixture of semiconducting SWNTs of different sizes which exhibit fluorescence at different wavelengths.

Single walled carbon nanotubes are sheets of graphene—single layer of graphite—rolled into a molecular cylinder and indexed by a vector connecting two points on the hexagonal lattice that conceptually forms the tubule with a given “chiral” twist. Hence, (n,m) nanotubes are those formed by connecting the hexagon with one n units across and m units down (n>m by convention). Carbon nanotubes show a relationship between geometric and electronic structure: the 1-D nature of the nanotube exerts a unique quantization the circumferential wave-vector and hence, simple perturbations of this chirality vector yield substantial changes in molecular properties. When |n−m|=0, the system is metallic in nature while if |n−m|=3q (with being q a nonzero integer) the nanotube possesses a small curvature induced gap and if |n−m|≠3q then the system is semiconducting with a measurable band-gap.

The sensing composition optionally contains SWNTs that are not semiconducting, i.e. metallic SWNTs, that are complexed with one or more proteins or other polymers, SWNTs (semiconducting or metallic) that are fully or partially complexed with proteins and/or polymers and/or surfactants, other carbon nanotubes or other carbon nanostructured materials that are complexed with protein (which may or may not be sensing proteins), polymers (which may or may not be sensing polymer) and/or surfactant, as well as aggregates, including ropes, of SWNTs, or aggregates of other carbon nanotubes or nanostructured materials. The sensing composition may further contain amorphous carbon and other byproducts of carbon nanotube synthesis, such as residual catalyst. Preferably, the types and levels of any of these optional components are sufficiently low to minimize detrimental effects on the function of the sensing composition.

Carbon nanotube/polymer complexes can be made by initial formation of individually dispersed carbon nanotubes. Individually dispersed nanotubes can be formed essentially as previously described by dispersion of carbon nanotube product in aqueous surfactant solution employing high-sheer mixing and sonication to disperse the nanotubes in surfactant, followed by centrifugation to aggregate bundles or ropes of nanotubes and decanting of the upper portion (e.g., 75-80%) of the supernatant to obtain micelle-suspended carbon nanotube solutions or dispersions (e.g., containing 20-25 mg/L of carbon nanotubes). Surfactant-dispersed carbon nanotubes are contacted with polymer solutions, preferably aqueous solutions of polymer, and subjected to dialysis under conditions in which the surfactant is removed without removal of the polymer or carbon nanotube. As surfactant is removed by dialysis, carbon nanotube/polymer complexes are formed.

The amount and type of surfactant employed for dispersion of carbon nanotubes can be readily determined employing methods that are well-known in the art. As noted in detail below, the surfactant employed must be compatible with the components of the sensing compositions, particularly with the sensing polymer, specifically with the sensing protein. The surfactant must not destroy the function of the sensing polymer or sensing protein. In certain cases, the surfactant must be a non-denaturing surfactant that does not significantly detrimentally affect the function (e.g., binding or enzymatic function) of the protein or other polymer. The amount of surfactant needed to disperse the carbon nanotubes can be determined by routine experimentation. It is preferred to employ the minimum amount of surfactant needed to provide individually dispersed carbon nanotubes. Surfactants are typically employed between about 0.1% to about 10% by weight. (more typically from 0.5% to 5% by weight) in aqueous solution to disperse carbon nanotubes.

For the formation of carbon nanotube/protein complexes, the surfactant originally employed to form the individually dispersed carbon nanotubes is replaced with a non-denaturing surfactant. For example, 1% by weight in water of sodium dodecylsulfate (SDS) can be replaced by 2% by weight in water of sodium cholate. Surfactant-dispersed carbon nanotubes are contacted in aqueous solution with functional protein or other polymer and subjected to dialysis under conditions in which the surfactant is removed without removal of the protein or carbon nanotube and the protein retains function. As surfactant is removed by dialysis, carbon nanotube/protein complexes are formed. The surfactant employed is of sufficiently low molecular weight to be removed by dialysis while the polymer is not.

Complexes of carbon nanotubes with sensing polymers can be prepared by methods other than the dialysis method specifically described herein. In some cases, the polymer may be complexed with the nanotube simply by contacting the nanotube with a sufficient amount of polymer and applying vigorous mixing (e.g., sonication), if necessary to obtain dispersed nanotubes. In other cases, an already dispersed nanotube composition comprising surfactant or polymer which functions for dispersion of the nanotube may be contacted with a sufficient amount of the sensing polymer and if necessary apply vigorous mixing to displace at least a portion of the surfactant or polymer already associated with the nanotube.

The preparation of surfactant dispersed carbon nanotubes employs vigorous mixing, for example high shear mixing, which may be provided using a high speed mixer, a homogenizer, a microfluidizer or other analogous mixing methods known in the art. Sonication, including various ultrasonication methods can be employed for dispersion. Preferred methods for dispersion involve a combination of high sheer mixing and sonication. See, for example, WO 03/050332 and WO 02/095099, each of which is incorporated by reference in its entirety.

In some embodiments, analyte sensing compositions include one or more carbon nanotube/protein complexes. In these complexes, one or more protein molecules are non-covalently associated with the carbon nanotube. Preferably, the protein molecule or molecules complexed with the carbon nanotube provide monolayer coverage or less of the carbon nanotube by protein. The complexed protein retains its biological function and the complexed carbon nanotube is a semi-conducting carbon nanotube which exhibits band gap fluorescence.

In some embodiments, analyte sensing compositions include one or more carbon nanotube/polymer complexes. In these complexes, one or more polymer molecules are non-covalently associated with the carbon nanotube. Preferably, the polymer molecule or molecules complexed with the carbon nanotube provide monolayer coverage or less of the carbon nanotube by polymer. The complexed polymer retains its biological function and the complexed carbon nanotube is a semi-conducting carbon nanotube which exhibits band gap fluorescence.

Non-denaturing surfactants include anionic surfactants, non-ionic surfactants and zwitterionic (or amphoteric) surfactants. The term denature (or denaturing) is used herein with respect to protein structure and function. A denatured protein is one that has lost its functional structure. Contact with surfactants, as well as other environmental changes (e.g., temperature or pH changes), can cause structural changes in proteins, and these structural changes can affect one or more of the biological functions of the protein. For example, a denatured enzyme will no longer exhibit enzymatic function. Contact with a non-denaturing surfactant does not have any significant detrimental effect on one or more of the biological functions of a given protein. A normally denaturing surfactant may function as a non-denaturing surfactant over a selected concentration range or with respect to certain proteins which are more resistant to its denaturing effect than most other proteins.

Non-denaturing surfactants include, among others, bile acids and derivatives of bile acids, e.g., cholate (salts of cholic acid, particularly sodium cholate), deoxycholate (salts of deoxycholic acid, particularly sodium deoxycholate), sulfobetaine derivatives of cholic acid, particularly 3-[(3-cholamidopropyl)-dimethylammonio]-1-propanesulfonate (CHAPS); carbohydrate-based surfactants, for example, alkyl glucosides, particularly n-alkyl-β-glucosides (more specifically, n-octyl-α-glucoside (OG)), alkyl thioglucosides, particularly n-alkyl-β-thioglucosides (more specifically, n-octyl-β-thioglucoside (OTG)); alkyl maltosides, particularly n-alkyl-β-maltosides (more specifically, n-dodecyl-β-glucoside); alkyl dimethyl amine oxides (e.g., (C₆-C₁₄) alkyldimethyl amine oxides, particularly lauryidimethyl amine oxide), non-ionic polyoxyethylene surfactants, e.g., Triton™ X-100 (or octyl phenol ethoxylate), Lubrol™ PX, Chemal LA-9 (polyoxyethylene(9)lauryl alcohol); and glycidols, e.g., p-sonomylphenoxypoly(glycidol) (Surfactant 10G). A normally non-denaturing surfactant may function as a denaturing surfactant over a selected concentration range or with respect to certain proteins which are more sensitive to its denaturing effect than most other proteins.

Non-denaturing surfactant can also include mixtures of non-denaturing surfactants with denaturing surfactant where the amount of denaturing surfactant is sufficiently low in the mixture to avoid detrimental effect on the protein. Denaturing of a protein by a given surfactant is dependent upon the concentration of surfactant in contact with the protein and may also depend upon other environmental conditions (temperature, pH, ionic strength, etc.) to which the protein is being subjected. The denaturing effects of a selected surfactant, at selected concentrations, upon a selected protein can be readily assessed by methods that are well-known in the art.

Surfactants preferred for use in the preparation of carbon nanotube complexes are dialyzable, i.e., capable of being selectively removed form a surfactant dispersed carbon nanotubes by dialysis without significant removal of carbon nanotubes or the polymers that are to be complexed with the carbon nanotubes. Dialyzable, non-denaturing surfactants include, among others, bile acids and derivatives of bile acids, e.g., cholate (salts of cholic acid, particularly sodium cholate), deoxycholate (salts of deoxycholic acid, particularly sodium deoxycholate), sulfobetaine derivatives of cholic acid, particularly 3-[(3-cholamidopropyl)-dimethylammonio]-1-propanesulfonate (CHAPS); carbohydrate-based surfactants, for example, alkyl glucosides, (e.g., C₆-C₁₄ alkyl glucosides), particularly n-alkyl-β-glucosides (more specifically, n-octyl-β-glucoside (OG)), alkyl thioglucosides, (e.g., C₆-C₁₄ alkyl thioglucosides), particularly n-alkyl-β-thioglucosides (more specifically, n-octyl-β-thioglucoside (OTG)); alkyl maltosides, (e.g., C₆-C₁₄ alkyl maltosides), particularly n-alkyl-3-maltosides (more specifically, n-dodecyl-β-glucoside); and alkyl dimethyl amine oxides (e.g., (C₆-C₁₄) alkyldimethyl amine oxides, particularly lauryldimethyl amine oxide). Dialyzable, non-denaturing surfactants for use in a given application with a given protein can be readily identified employing well-known methods.

The term protein is used herein as broadly as it is in the art to refer to molecules of one or more polypeptide chains which may be linked to each other by one or more disulfide bonds. Proteins include glycoproteins (proteins linked to one or more carbohydrates), lipoproteins (proteins linked to one or more lipids), metalloproteins (proteins linked to one or more metal ions) and nucleoproteins (proteins linked to one or more nucleic acids). The term protein is however intended to exclude small peptides, such as those having less than 50 amino acids. The term protein includes polypeptides having 50 or more amino acids. A protein may comprise one or more subunits and the subunits may be the same or different. For example, a protein may be a homodimer (having two subunits that are the same) or a heterodimer (having two subunits that are different). Proteins typically have one or more biological functions. Proteins include enzymes which catalyze reactions and antibodies, transport proteins, receptor proteins or other proteins which bind to other chemical species (peptides, nucleic acids, carbohydrates, lipids, other proteins, antigens, haptens, etc.). Proteins useful in sensing compositions include soluble proteins, membrane proteins and transmembrane proteins. Soluble proteins are of particular interest for the formation of carbon nanotube/protein complexes.

The term polypeptide is used to refer to peptides having 20 or more amino acids and in particular. Peptides such as those reported in WO 03/102020, which is incorporated by reference in its entirety, are optionally excluded from the meaning of the term polypeptide as used herein.

Useful proteins include those that exhibit selective binding to given chemical species or, which are one member of a set (particularly a pair) of binding partners (e.g., avidin and biotin, a receptor and a receptor ligand, or an antibody or antibody fragment and an antigen to which it binds). In specific embodiments, useful proteins include soluble receptors and cell surface receptors. In other specific embodiments, useful proteins include G-protein coupled receptors (GPCRs). In more specific embodiments, useful proteins include steroid receptors, particularly estrogen receptors.

In some embodiments, proteins useful in sensing compositions may contain one or more of the carbon nanotube binding sequences disclosed in WO 03/102020, but in other embodiments, proteins useful in sensing compositions do not contain any one or more of the carbon nanotube binding sequences disclosed in WO 03/102020.

Enzymes function by binding to a substrate and catalyze a reaction of the substrate. Substrate selectivity or specificity of an enzyme is, at least in part, determined by the selectivity or specificity with which the enzyme binds to a substrate. Enzymes include among others those that catalyze oxidation and/or reduction reactions and those that catalyze cleavage of certain bonds or the formation of certain bonds. It is understood in the art that enzyme function may require the presence of cofactors and/or co-enzymes. Further, it is understood in the art that enzyme function may be affected by pH, ionic strength, temperature or the presence of inhibitors. Methods and devices as described herein can employ enzymes which are well-known in the art so that the requirements for any co-factors and/or co-enzymes and the effect of pH, ionic strength, temperature and other environmental factors as well as potential inhibitors will also be well-known. Enzymes useful in sensing compositions include oxidases, dehyrogenases, esterases, oxigenases, lipases, and kinases, among others which may be obtained from various sources. More specifically, enzymes useful in analyte sensing compositions include glucose oxidases, glucose dehydrogenases, galactose oxidases, glutamate oxidases, L-amino acid oxidases, D-amino acid oxidases, cholesterol oxidases, cholesterol esterases, choline oxidases, lipoxigenases, lipoprotein lipases, glycerol kinases, glycerol-3-phosphate oxidases, lactate oxidases, lactate dehydrogenases, pyruvate oxidases, alcohol oxidases, bilirubin oxidases, sarcosine oxidases, uricases, and xanthine oxidases and wherein the analyte is a substrate for the enzyme.

Proteins useful in sensing compositions may be truncations, variants, derivatives, or semi-synthetic analogs of a naturally-occurring protein which, for example, has been modified by modification of one or more amino acids to exhibit altered biological function, e.g., altered binding, compared to the naturally-occurring protein, is a deglycosylated form of a naturally-occurring protein or a variant or derivative thereof, or has glycosylation different than that of a naturally-occurring protein. Proteins as well as protein truncations, variants, fusions, derivatives or semi-synthetic analogs of naturally-occurring proteins and enzymes, exhibit a biological function that can be used detect an analyte. Protein truncations, variants, fusions, derivatives or semi-synthetic analogs of naturally-occurring proteins and enzymes may exhibit altered binding affinity and/or altered biological function compared to naturally-occurring forms of the proteins. Protein truncations, for example, specifically include the soluble portion or portions of membrane or transmembrane proteins. Protein fusions, for example, specifically include fusions of the soluble portion or portions of membrane or transmembrane proteins with soluble carrier proteins (or polypeptides).

Enzymes useful in sensing compositions may be a truncation, variant, fusion, derivative, or semi-synthetic analog of a naturally-occurring enzyme which, for example, has been modified by modification of one or more amino acids to exhibit altered activity, e.g., enhanced activity, compared to the naturally-occurring enzyme, is a deglycosylated form of a naturally-occurring enzyme or a variant, fusion, or derivative thereof, has altered glycosylation than that of a naturally-occurring enzyme, is formed by reconstitution of an apo-enzyme with its required co-factor (e.g., FAD), is formed by reconstitution of an apo-enzyme with a derivatized co-factor. Enzyme variants, fusions, derivatives or semi-synthetic analogs of naturally-occurring enzymes may exhibit altered substrate specificity and/or altered enzyme kinetics compared to naturally-occurring forms of the enzyme.

The term antibody (or immunoglobulin) as used herein is intended to encompass its broadest use in the art and specifically refers to any protein or protein fragment(s) that function as an antibody and is specifically intended to include antibody fragments including, among others, Fab′ fragments. Antibodies are proteins synthesized by an animal in response to a foreign substance (antigen or hapten) which exhibit specific binding affinity for the foreign substance. The term antibody includes both polyclonal and monoclonal antibodies. Polyclonal and monoclonal antibodies selective for a given antigen are readily available from commercial sources or can be routinely prepared using methods and materials that are well-known in the art. A monoclonal antibody preparation can be derived from techniques involving hybridomas and recombinant techniques. Various expression, preparation, and purification methodologies can be used as known in the art. For example, microbial expression of antibodies can be employed (e.g., see U.S. Pat. No. 5,648,237). Human, humanized, and other chimeric antibodies can be produced using methods well-known in the art.

Sensing compositions can include carbon nanotube complexes with polymers, particularly sensing polysaccharides. The term polysaccharide is used generally herein to include polymers of any monosaccharide or combination of monosaccharides. A polysaccharide typically contains 20 or more monosaccharide units. Oligosaccharide containing less than 20 monsaccharide units can be used if they are found to complex with carbon nanotubes. For assays for monosaccharide analytes, polymers of the monosaccharide analyte (e.g., polymers of glucose for use in assays for glucose) may be used. Polysaccharides and oligosaccharides can be derivatized with one or more chemically selective groups or moieties to impart chemical selectively to the polysaccharide.

Sensing compositions can include carbon nanotube complexes with derivatized polymers that are not proteins, polysaccharides (or oligosaccharides) or other biological polymers such as polynucleotides. Polymers which complex to carbon nanotubes and are useful in sensing compositions and methods herein include polymers which are derivatized to contain one or more chemically selective groups or moieties which impart chemical selectively to the polymer. Polymers that can be usefully derivatized include poly(ethylene glycol), poly(vinyl alcohol), poly(vinyl chloride), (e.g., and copolymers thereof, and polysorbitan esters (e.g., polyoxyethylene sorbitan fatty acid esters.)

A sensing element for detecting an analyte can include a selectively porous container adapted for receiving and retaining the components of a sensing composition. The container is sufficiently porous to allow analyte to enter the container without allowing the functional components of the analyte sensing composition to exit the container. The sensing composition is dispersed in a liquid or solid material. Typical liquids are aqueous solutions which include solutions in which the majority component is water, but which may include alcohols, glycols and related water soluble materials that do not affect the ability of the sensing composition to detect or quantitate analyte. The sensing composition may be dispersed in a solid matrix. The matrix can be formed from various polymers, silica, quartz or other glass, ceramics and metals with the proviso that the metal matrix is insulated from the surface with a coating that preserved the optical properties of the carbon nanotube/sensing polymer complexes. The matrix can be formed from a combination of such solid materials. The matrix can also be a semi-solid material such as a gel or a paste. The matrix must be sufficiently porous to allow analyte to enter without loss of sensing composition components that are needed to analyte detection. The matrix must also be sufficiently optically thin or transparent to the excitation and emission to allow detection of analytes. A solid matrix with dispersed sensing composition can serve as a sensing element. In a preferred embodiment, the sensing element is an implantable container or matrix comprising sensing composition which is biocompatible. The term “biocompatible” is employed as broadly as the term is used in the art and in preferred embodiments for human or veterinary applications the term refers to materials that cause minimal irritation and/or allergic response on implantation. The term also preferably refers to materials in which biofouling of pores is minimized.

Sensing elements include those that are implantable in tissue. Such sensors may be affected by foreign body encapsulation and/or membrane biofouling of the sensor surface. Fibroblast encapsulation at the site of sensor element implantation has been reviewed and art-recognized solutions to this problem include administration of antigenic factors and anti-inflammatory pharmaceuticals at the site of implantation to promote neovascularization. A sensor surface may be biofouled as endothelial cells adhere and either block or in some cases consume analyte, thus decreasing the accuracy or otherwise decreasing or destroying the function of the sensor. Sensor architecture can play a significant role in exacerbating or ameliorating the biofouling problem. Biofouling can limit the flux of analyte to the sensor as cellular adhesion becomes more pronounced. Electrochemical sensors, which are the most widely employed for glucose detection, measure the flux of analyte (e.g., glucose) from a limiting membrane. Biofouling in such sensors can decrease the measured signal and is corrected only by frequent recalibration and eventually replacement is required. In contrast, optical sensors, measure the concentration of analyte at the sensor directly and fouling results in a delay in sensor response. A sensor that measures concentrations of analyte directly does not exhibit significant distortion of the measured analyte concentration until the sensor response rate becomes commensurate with the rate of change in the bulk. Implanted optical sensors will exhibit an increased stability and longer useful life on implantation compared to sensors which measure analyte flux such as electrochemical sensors.

A sensing system for detecting one or more analytes comprises one or more sensing elements and a detector for measuring an optical response of the complexes in the sensing solution. Any appropriate optical detector may be employed. The detector can include any and all necessary device elements for detecting light and converting the signal detected into a form appropriate for analysis or display. Detectors and device elements for any needed signal conversion, analysis and display are known in the art and readily available for use. It is noted that the sensing elements of the system may be remote from the detector. More specifically, the sensing system can include a source of electromagnetic radiation to provide electromagnetic radiation of appropriate wavelength for exciting luminescence of the complexed carbon nanotube in the sensing composition which can be detected by the detector. Any known source appropriate for the sensor application can be employed including light emitting diodes, or lasers. It is noted that the excitation source may be remote from the sensor and may also be remote from the detector. In a specific embodiment, the detector and the excitation source may be combined in a single device. Those of ordinary skill in the art can select light sources and/or detectors appropriate for use in sensor systems in view of what is generally known in the art and the specific wavelengths or wavelength ranges in which the sensor is to operate.

Non-limiting examples of analytes that can be determined using the compositions and methods described herein include specific proteins, viruses, hormones, drugs, nucleic acids and polysaccharides; specifically antibodies, e.g., IgD, IgG, IgM or IgA immunoglobulins to HTLV-I, HIV, Hepatitis A, B and non A/non B, Rubella, Measles, Human Parvovirus B 19, Mumps, Malaria, Chicken Pox or Leukemia; human and animal hormones, e.g., thyroid stimulating hormone (TSH), thyroxine (T4), luteinizing hormone (LH), follicle-stimulating hormones (FSH), testosterone, progesterone, human chorionic gonadotropin, estradiol; other proteins or peptides, e.g. troponin I, c-reactive protein, myoglobin, brain natriuretic protein, prostate specific antigen (PSA), free-PSA, complexed-PSA, pro-PSA, EPCA-2, PCADM-1, ABCA5, hK2, beta-MSP (PSP94), AZGP1, Annexin A3, PSCA, PSMA, JM27, PAP; drugs, e.g., paracetamol or theophylline; marker nucleic acids, e.g., PCA3, TMPRS-ERG; polysaccharides such as cell surface antigens for HLA tissue typing and bacterial cell wall material. Chemicals that may be detected include explosives such as TNT, nerve agents, and environmentally hazardous compounds such as polychlorinated biphenyls (PCBs), dioxins, hydrocarbons and MTBE. Analytes may be detected in a wide variety of sample types, including a liquid sample or solid sample, a biological fluid, an organism, a microorganism or medium containing a microorganism, an animal, a mammal, a human, a cell line or medium containing a cell line. Typical sample fluids include physiological fluids such as human or animal whole blood, blood serum, blood plasma, semen, tears, urine, sweat, saliva, cerebro-spinal fluid, vaginal secretions; in-vitro fluids used in research or environmental fluids such as aqueous liquids suspected of being contaminated by the analyte. In some embodiments, one or more of the above-mentioned reagents is stored in a channel or chamber of a fluidic device prior to first use in order to perform a specific test or assay. In some embodiments, the sample can be cancer cells. In other embodiments, the sample can be fermentation cells, incubation cells, generation cells, or biofuel cells.

As used herein, the terms “determination” or “determining” generally refer to the analysis of a species or signal, for example, quantitatively or qualitatively (whether the species or signal is present and/or in what amount or concentration), and/or the detection of the presence or absence of the species or signals. “Determination” or “determining” may also refer to the analysis of an interaction between two or more species or signals, for example, quantitatively or qualitatively, and/or by detecting the presence or absence of the interaction. For example, the method may include the use of a device capable of producing a first, determinable signal (e.g., a reference signal), such as an electrical signal, an optical signal, or the like, in the absence of an analyte. The device may then be exposed to a sample suspected of containing an analyte, wherein the analyte, if present, may interact with one or more components of the device to cause a change in the signal produced by the device. Determination of the change in the signal may then determine the analyte.

Specific examples of determining a species or signal include, but are not limited to, determining the presence, absence, and/or concentration of a species, determining a value or a change in value of a wavelength or intensity of electromagnetic radiation (e.g., a photoluminescence emission), determining the temperature or a change in temperature of a composition, determining the pH or a change in pH of a composition, and the like.

In one embodiment, a sensing composition includes a complex of a SWNT with a sensing polymer which includes an organic polymer modified with analyte-binding protein. The modification can be non-covalent (e.g., a non-covalent association of the organic polymer with the analyte binding protein) or covalent (e.g., the organic polymer is covalently bound to the analyte binding protein). The organic polymer can be, e.g., a carboxylated poly(vinyl alcohol) (cPVA).

The analyte binding protein can be one that undergoes a substantial conformational change when binding the analyte. For example, members of the periplasmic binding protein family can undergo a substantial conformational change when binding an analyte. The analyte binding protein can be a monosaccharide binding protein, e.g., glucose binding protein (GBP). GBP is an example of a periplasmic binding protein that undergoes a substantial conformational change when binding an analyte.

Thus, the sensing polymer can be cPVA covalently modified with GBP. GBP is a periplasmic binding protein which binds glucose with a high degree of specificity. GBP exhibits equilibrium binding kinetics; in other words, glucose can be easily unbound from a glucose-GBP complex, thus providing for a reversible binding event. See, for example, U.S. Patent Application Publication no. 2010/0279421, which is incorporated by reference in its entirety.

High throughput analysis methods, where libraries of homologous molecules are screened and compared for efficacy, can be valuable for drug discovery and catalytic development. The application of high throughput analysis methods to the problem of optical sensor development can provide structural and chemical clues as to the most effective ways of transducing analyte binding to optically modulate SWNTs. For example, a library of boronic acid (BA) constructs to sodium cholate suspended SWNTs (SC/SWNTs) can be screened for their ability to modulate fluorescence emission in response to glucose. An examination of successful candidates can yield structural and chemical design rules to enable such sensors.

A boronic acid can be an excellent molecular receptor for saccharides. The detection and monitoring of saccharides (e.g., glucose and fructose) can be vital in medical diagnostics, biomedical research, and biotechnology. Boronic acids have attracted attention as an alternative receptor to enzymes for saccharide detection (e.g., glucose oxidases for glucose detection). The enzyme-based sensing has the disadvantages that since it is based on the rate of the reaction between the enzyme and the analyte, this approach can be sensitive to various factors that affect the enzyme activity and the mass transport of the analyte, it can consume the analyte, and it can require mediators; in contrast, the boronic acid-based sensing can be based on the reversible and equilibrium-based complexation of boronic acids and saccharides, thus consuming no analytes.

The reversible complexation of saccharides with aromatic boronic acids can produce a stable boronate anion, changing the electronic properties of the boronic acids, such as the reduction potential of aromatic boronic acids. This alternation in the electronic properties of aromatic boronic acids upon binding of saccharides has been a basic scheme for various boronic acid-based saccharide sensing approaches, including electrochemical, fluorescence, and colorimetric measurements. Thus, complexation of saccharides with aromatic boronic acids conjugated on the surface of SWNTs, for example, through η-η interactions between the graphene sidewall of SWNTs and the aromatic moiety of the boronic acids, can modulate the SWNT fluorescence signal in response to binding of saccharides.

In one embodiment, a sensing composition includes a complex of a SWNT with a sensing polymer which includes an organic polymer modified with a chemical moiety that is capable of reacting with an analyte. The modification can be non-covalent (e.g., a non-covalent association of the organic polymer with the reactive moiety) or covalent (e.g., the organic polymer is covalently bound to the reactive moiety). The reactive moiety can be a boronic acid, and the analyte can be a monosaccharide, e.g., glucose. The organic polymer can include diol groups, such that a boronic acid forms a boronate ester with the organic polymer. In this configuration, when analyte molecules are introduced to the system, they bind to the boronic acid, detaching it from the organic polymer. Thus the analyte competes with the organic polymer for the binding of the boronic acid; the fluorescence change resulting from the detachment of the boronic acid is used to measure the analyte. Alternatively, the organic polymer can be a surfactant (e.g., dextran, PVA, chitosan, alginate, and lipid PEG) modified such that the boronic acid is exposed toward the solution to facilitate binding with the analyte. In this configuration, the binding of analyte molecules to the boronic acid modulates the fluorescence of the SWNT. See, e.g., U.S. Patent Application Publication no. 2010/0279421, U.S. patent application Ser. No. 13/090,199, filed Apr. 19, 2011, and provisional application No. 61/325,599, filed Apr. 19, 2010, each of which is incorporated by reference in its entirety.

In another embodiment, a sensing composition includes a complex of a SWNT with a boronic acid (BA-SWNT complex). The fluorescence of BA-SWNT complexes, quenched by the attachment of boronic acids to nanotubes, can be selectively recovered in response to the binding of glucose in the physiological range of glucose concentrations. The reversible fluorescence quenching of the BA-SWNT complex that exploits boronic acids as a molecular receptor can provide SWNT-based highly stable and sensitive, nIR optical sensing of saccharides. The optical sensing of glucose holds promise for noninvasive in vivo continuous glucose monitoring, important for diabetes management. For instance, commercial noninvasive continuous glucose monitors for long-term use are not currently available. With the non-photobleaching, nIR fluorescence of SWNTs, the SWNT-based nIR optical sensing of glucose has great potential in this regard.

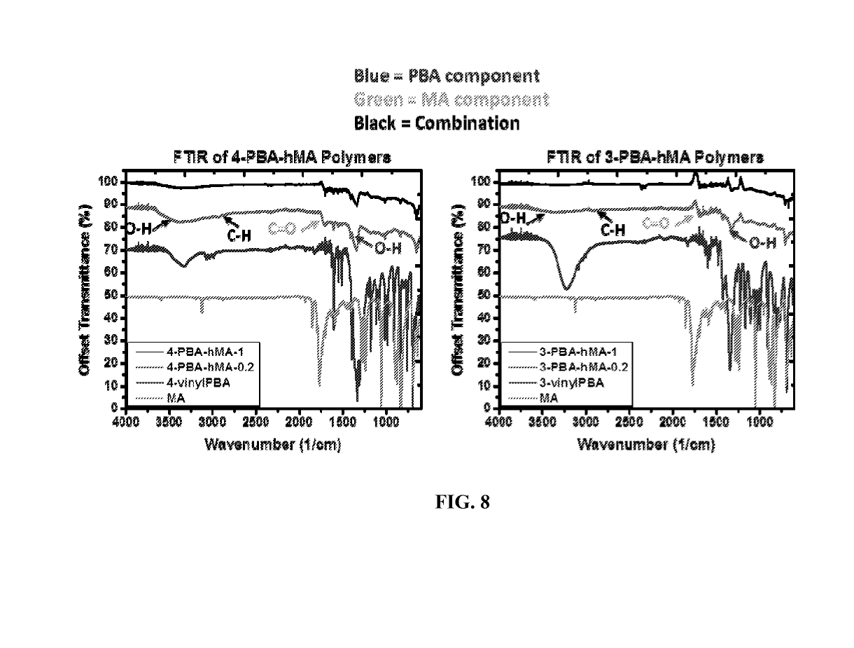

The modulation of SWNT fluorescence of SWNT through the binding of analyte molecules to boronic acid results from either (i) the shift of the peak wavelength or (ii) the change in the fluorescence intensity. Depending on the boronic acid used, the fluorescence intensity can be increased or decreased upon the binding of analyte molecules to a boronic acid-SWNT sensor. For example, when using 4-chlorophenylboronic acid, the fluorescence intensity can decrease in the presence of glucose. In contrast, the fluorescence intensity of the sensor can increase upon exposure to glucose when using 4-cyanophenylboronic acid (see FIGS. 8A-8C). The shift of the peak and/or the change of the fluorescence intensity can thus be used to measure an analyte. Glucose recognition and transduction can be facilitated by para-substituted, electron withdrawing phenyl boronic acids that are sufficiently hydrophobic as to adsorb to the nanotube surface.

In general, any boronic acid or boronate ester moiety containing monomers can be incorporated into the sensing polymer. A boron-containing moiety can be a boronic acid, a borinic acid, or a boronic acid ester. Examples of such groups are —B(OH)₂, —B(OH)(OR) and —B(OR)(OR′) in which R and R′ are alkyl groups of from 1 to 6 carbon atoms which, in some embodiments, can be linked together to form a cyclic ester. In some embodiments, the boronic acids can be an aryl boronic acid, particularly a vinyl aryl boronic acid, such as 3-vinylphenylboronic acid (3vPBA) and 4-vinylphenylboronic acid (4vPBA) or its positional isomers. Other substituted aryl boronic acids containing a polymerizable functional group (e.g., an alkene) and optional functionality on the aryl ring (e.g., alkyl groups, halogens, carbonyl groups, amines, hydroxyl groups, carboxylic acids and their derivatives, and the like) can also be used. In other embodiments, the boronic acids moiety containing a polymerizable functional group can be alkyl, alkenyl, or alkynyl boronic acids (i.e., aliphatic boronic acids) in which the alkyl, alkenyl, or alkynyl groups can contain optional substitution.

In another embodiment, a sensing composition can be encapsulated in a microparticle, e.g., a hydrogel microparticle. The microparticle can be biocompatible and of an injectable size, e.g., 50 to 500 μm. The hydrogel microparticle can have a microbead structure or a core-shell structure. In a microbead structure, the microbeads contain the sensing composition dispersed in the hydrogel structures. In a core-shell (or microcapsule) structure, the microparticle includes an aqueous core solution of the sensing composition (e.g., in PBS), and the hydrogel shell surrounding the aqueous core solution. Various biocompatible hydrogels, such as alginate, PEG, and chitosan, can be used for both the microbeads and the core-shell microparticles. The hydrogel microparticles confine and protect the sensing composition, while allowing analytes (e.g., glucose) to freely diffuse into and out of the hydrogel microparticles. These hydrogel microparticles can be subcutaneously implanted with minimal invasiveness, and reduce biofouling, which is favorable for long-term, accurate biosensor performance. The hydrogel microparticles can be produced using commercially available encapsulating systems (e.g., encapsulating systems from Inotech and Nisco) and flow-focusing microfluidic devices.

Nanomaterial based sensors have demonstrated the ability to impact a variety of applications. In particular, single-walled carbon nanotubes (SWNT) have been used in sensing a range of biological and chemical media for personal safety as well as for diagnostics. See, Endo, M., M. S. Strano, and P. M. Ajayan, Potential applications of carbon nanotubes. Carbon Nanotubes, 2008. 111: p. 13-61, McNicholas, T. P., et al., Sensitive Detection of Elemental Mercury Vapor by Gold-Nanoparticle-Decorated Carbon Nanotube Sensors. Journal of Physical Chemistry C. 115(28): p. 13927-13931, Yoon, H., et al., Chemical approaches to glucose detection using the near-infrared fluorescence from single-walled carbon nanotubes. Abstracts of Papers of the American Chemical Society. 240, Barone, P. W., R. S. Parker, and M. S. Strano, In vivo fluorescence detection of glucose using a single-walled carbon nanotube optical sensor: Design, fluorophore properties, advantages, and disadvantages. Analytical Chemistry, 2005. 77(23): p. 7556-7562, Boghossian, A. A., et al., Near-Infrared Fluorescent Sensors based on Single-Walled Carbon Nanotubes for Life Sciences Applications. Chemsuschem, 2011. 4(7): p. 848-863, and Liu, Z., et al., Carbon Nanotubes in Biology and Medicine: In vitro and in vivo Detection, Imaging and Drug Delivery. Nano Research, 2009. 2(2): p. 85-120, each of which is incorporated by reference in its entirety. Their unique structure consists of a single layer of carbon atoms formed into a tubular construct. Individual SWNT are therefore comprised exclusively of surface bound carbon atoms which are exposed to the surrounding media. SeeMu, B., et al., A Structure-Function Relationship for the Optical Modulation of Phenyl Boronic Acid-Grafted, Polyethylene Glycol-Wrapped Single-Walled Carbon Nanotubes. Journal of the American Chemical Society, 2012. 134(42): p. 17620-17627, Barone, P. W., et al., Modulation of Single-Walled Carbon Nanotube Photoluminescence by Hydrogel Swelling. Acs Nano, 2009. 3(12): p. 3869-3877, and Chen, J., et al., Effect of Surfactant Type and Redox Polymer Type on Single-Walled Carbon Nanotube Modified Electrodes. Langmuir, 2013. 29(33): p. 10586-10595, each of which is incorporated by reference in its entirety. This aspect, combined with their unique electronic band structure has made them ideal materials for many electrochemical and optochemical sensing applications. See Kong, J., et al., Nanotube molecular wires as chemical sensors. Science, 2000. 287(5453): p. 622-625, and Barone, P. W. S., M. S., Single Walled Carbon Nanotubes as Reporters for the Optical Detection of Glucose. Journal of Diabetes Science and Technology, 2009. 3(2): p. 11, each of which is incorporated by reference in its entirety. Additionally, SWNT fluoresce in the near infrared (nIR) region of the photospectra. This is an important fact when considering materials for implantable sensor applications as this fluorescence occurs in a window between where water and blood absorb. See, Yum, K., et al., Single-walled carbon nanotube-based near-infrared optical glucose sensors toward in vivo continuous glucose monitoring. Journal of diabetes science and technology, 2013. 7(1): p. 72-87, which is incorporated by reference in its entirety. Thus, this optical signal can transmit through biological media. Furthermore, unlike other traditional organic flourophores, SWNT do not photobleach. This fact allows SWNT sensors to report fluorescent data over unparalleled lengths of time.

Diabetes Mellitus presently affects 347 million patients worldwide as of 2013. Furthermore, the World Health Organization (WHO) projects it to be the 7^{th} leading cause of death worldwide by 2030. Type 1 diabetics suffer from deficient insulin production which does not allow the patient to appropriately regulate excessively high blood glucose levels, known as hyperglycemia; these patients also suffer from inabilities to regulate low blood glucose levels, known as hypoglycemia. Type 2 diabetics suffer from an inability to efficiently utilized insulin. Regardless of the type, diabetics may suffer from kidney failure, cardiac disease, blindness, nerve damage leading to limb amputation and even death. Appropriate regulation of blood glucose levels have been suggested to help minimize the potentially fatal side effects of diabetes. See Center_for_Disease_Control_Diabetes_Fact_Sheet, National Diabetes Fact Sheet 2011. 2011, World_Health_Organization_Diabetes_Fact_Sheet, World Health Organization Diabetes Fact Sheet, 2013, Seissler, J., Blood glucose control in type 2 diabetes. Internist, 2007. 48(7): p. 676-+, Mauras, N., et al., Continuous glucose monitoring in type 1 diabetes. Endocrine, 2013. 43(1): p. 41-50, and Hortensius, J., et al., What do professionals recommend regarding the frequency of self-monitoring of blood glucose? Netherlands Journal of Medicine, 2012. 70(6): p. 287-291, each of which is incorporated by reference in its entieryt. As such, continuous blood glucose monitoring may help patients avoid complications which can arise from “black out periods” between single point measurements. These single-point measurements, such as those in finger-prick based electrochemical detection methods, are currently the standard used by most patients. Continuous blood glucose monitors presently on the market include transdermal implants which are associated with open wounds. These open wounds can lead to biofouling and infection. See, Wickramasinghe, Y., Y. Yang, and S. A. Spencer, Current problems and potential techniques in in vivo glucose monitoring. Journal of Fluorescence, 2004. 14(5): p. 513-520, and Barone, P. W. and M. S. Strano, Single walled carbon nanotubes as reporters for the optical detection of glucose. Journal of diabetes science and technology, 2009. 3(2): p. 242-52, each of which is incorporated by reference in its entirety. Additionally, these and other products suffer from relatively short lifetimes. Typical sensor lifetimes range from 3-7 days before significant sensor attention or replacement is needed.

Previously, groups including our own have demonstrated examples of glucose sensors based on glucose oxidase (GOx) and glucose binding proteins (GBP). See Barone, P. W., et al., Near-infrared optical sensors based on single-walled carbon nanotubes. Nature Materials, 2005. 4(1): p. 86-U16, Tsai, T.-W., et al., Adsorption of Glucose Oxidase onto Single-Walled Carbon Nanotubes and Its Application in Layer-By-Layer Biosensors. Analytical Chemistry, 2009. 81(19): p. 7917-7925, and Yoon, H., et al., Periplasmic Binding Proteins as Optical Modulators of Single-Walled Carbon Nanotube Fluorescence: Amplifying a Nanoscale Actuator. Angewandte Chemie-International Edition. 50(8): p. 1828-1831, each of which is incorporated by reference in its entirety. While GOx remains a highly useful and robust system, implantable sensors based on GOx are limited by the production of hazardous H₂O₂ and the conversion of glucose to D-glucono-δ-lactone. GBP-based sensors demonstrated impressive selectivity and reversibility. However, there remains an opportunity to improve the magnitude of the glucose response. Furthermore, the robustness of the sensor may be improved by changing the glucose binding site from a protein to a small molecule. See, McNicholas, T. P., et al., Structure and Function of Glucose Binding Protein-Single Walled Carbon Nanotube Complexes. Small. 8(22): p. 3510-3516, which is incorporated by reference in its entirety. Several groups have sought to utilize the well-known interaction of boronic acids with saccharides to create glucose responsive systems. See, Hansen, J. S., et al., Arylboronic acids: A diabetic eye on glucose sensing. Sensors and Actuators B-Chemical, 2012. 161(1): p. 45-79, Billingsley, K., et al., Fluorescent Nano-Optodes for Glucose Detection. Analytical Chemistry, 2010. 82(9): p. 3707-3713, and Oh, W. K., et al., Fluorescent boronic acid-modified polymer nanoparticles for enantioselective monosaccharide detection. Analytical Methods, 2012. 4(4): p. 913-918, each of, which is incorporated by reference in its entirety. Billingsley et. al. used a competitive binding of the fluorophore alazirin red (ARS) and glucose to a free boronic acids to create glucose responsive microcapsules. When bound to the boronic acids, the ARS fluoresces visible light (˜495 nm). Addition of glucose causes the boronic acid-ARS binding equilibrium to shift to an unbound state; as a result, the ARS fluorescence is diminished. These microcapsules were successfully implanted in a mouse model where in vivo glucose detection was demonstrated over approximately one hour. However, using this fluorophore, issues of photobleaching over continuous probing may still be a limiting factor. Furthermore, this system relies on the binding energy of free boronic acids to saccharides. As a result, fructose is likely to be a significant interferant for in vivo glucose detection, as it binds strongest to free boronic acids. See, Savsunenko, O., et al., Functionalized Vesicles Based on Amphiphilic Boronic Acids: A System for Recognizing Biologically Important Polyols. Langmuir, 2013. 29(10): p. 3207-3213, which is incorporated by reference in its entirety.

Disclosed herein is a unique interaction between phenylboronic acid (PBA) derivatives and SWNT. See, Mu, B., et al., A Structure-Function Relationship for the Optical Modulation of Phenyl Boronic Acid-Grafted, Polyethylene Glycol-Wrapped Single-Walled Carbon Nanotubes. Journal of the American Chemical Society, 2012. 134(42): p. 17620-17627, and Yum, K., et al., Boronic Acid Library for Selective, Reversible Near-Infrared Fluorescence Quenching of Surfactant Suspended Single-Walled Carbon Nanotubes in Response to Glucose. Acs Nano, 2012. 6(1): p. 819-830, each of which is incorporated by reference in its entirety. Specifically, PBA adsorb to the surface of the SWNT in a π-π stacking mechanism, causing a fluorescent quenching of the SWNT. This results from an excited-state electron transfer from the SWNT to the PBA dopant level.^{[15]} Upon the addition of glucose, the resulting diol bond formation between the boronic acid and the glucose modulates the reduction potential or this PBA dopant level. This modulation occurs such that the excited state electron transfer between the SWNT and the PBA is either discouraged (turn-on response) or encouraged (turn-off response, or quenching). This allowed for the modulation of the SWNT fluorescent signal to act as a reporter for the addition of glucose. However, in this example, the individual PBA molecules were simply adsorbed to the surface of the SWNT, which were suspended using sodium cholate (SC). SC and similar surfactants suspend SWNT with a continuous adsorption and desorption from the SWNT surface. See, Hilmer, A. J., et al., Role of Adsorbed Surfactant in the Reaction of Aryl Diazonium Salts with Single-Walled Carbon Nanotubes. Langmuir, 2012. 28(2): p. 1309-1321, Bachilo, S. M., et al., Structure-assigned optical spectra of single-walled carbon nanotubes. Science, 2002. 298(5602): p. 2361-2366, Strano, M. S., et al., The role of surfactant adsorption during ultrasonication in the dispersion of single-walled carbon nanotubes. Journal of Nanoscience and Nanotechnology, 2003. 3(1-2): p. 81-86, and Usrey, M. L. and M. S. Strano, Controlling Single-Walled Carbon Nanotube Surface Adsorption with Covalent and Noncovalent Functionalization. Journal of Physical Chemistry C, 2009. 113(28): p. 12443-12453, each of which is incorporated by reference in its entirety. Because of this fact, dropping the surfactant concentration below the critical micelle concentration causes SWNT aggregation and therefore SWNT fluorescence quenching. Furthermore, most surfactants are not biocompatible, causing significant protein denaturation and other serious biological side effects. See, Howett, M. K., et al., A broad-spectrum microbicide with virucidal activity against sexually transmitted viruses. Antimicrobial Agents and Chemotherapy, 1999. 43(2): p. 314-321, which is incorporated by reference in its entirety. Also, individual PBA molecules may desorb from the SWNT surface over extended periods. As a result, there exists a critical need to develop a class of PBA-based polymers which can be directly used for suspending SWNT and which impart enhanced sensitivity and stability to the resulting nanosensor. Additionally, this polymer must enable the resulting nanosensor to respond quickly and selectively to the addition of saccharide analytes.

Reversible addition forward chain transfer (RAFT) polymerization is a highly versatile tool that allows the size and composition of polymers to be highly controlled and tuned. See, Henry, S. M., et al., pH-responsive poly(styrene-alt-maleic anhydride) alkylamide copolymers for intracellular drug delivery. Biomacromolecules, 2006. 7(8): p. 2407-2414, Cambre, J. N., et al., Facile strategy to well-defined water-soluble boronic acid (co)polymers. Journal of the American Chemical Society, 2007. 129(34): p. 10348-+, and Roy, D., J. N. Cambre, and B. S. Sumerlin, Sugar-responsive block copolymers by direct RAFT polymerization of unprotected boronic acid monomers. Chemical Communications, 2008(21): p. 2477-2479, each of which is incorporated by reference in its entirety. It has been used to create a variety of polymers for drug release and analyte detection. Henry et. al. used RAFT polymerization to create a polystyrene-alt-maleic anhydride polymer with pH dependent hemolytic activity, useful in intracellular drug delivery. See, Henry, S. M., et al., pH-responsive poly(styrene-alt-maleic anhydride) alkylamide copolymers for intracellular drug delivery. Biomacromolecules, 2006. 7(8): p. 2407-2414, which is incorporated by reference in its entirety. Roy and Sumerlin used RAFT to produce an aggregation-based sensor for both changes in pH and glucose addition. See, Roy, D. and B. S. Sumerlin, Glucose-Sensitivity of Boronic Acid Block Copolymers at Physiological pH. Acs Macro Letters. 1(5): p. 529-532, which is incorporated by reference in its entirety. It was based on a PBA component which either adopted a formal charge based on increase in the pH, or coupled to glucose, to impart water stability.

Disclosed herein is a nanosensor comprised of a novel composition of phenylboronic acid polymer complexed with single-walled carbon nanotubes (SWNT). This polymer is formed using reversible addition forward chain transfer polymerization and is used to impart water solubility and saccharide sensitivity to individual SWNT fluorophores. One such polymer-SWNT nanosensor demonstrates a SWNT nIR fluorescent signal modulation of −12.64±0.722% when exposed to 10 mM glucose. Furthermore, this sensing mechanism is confirmed as occurring nearly instantaneously, as is demonstrated by transient measurements. The selectivity of these complexes is distinct from that of free boronic acid moieties. Furthermore, polymers having different phenylboronic acid derivatives and molecular weights impart distinct saccharide binding profiles when coupled to SWNT. As such, this complex represents an intriguing new class of saccharide sensors which may be utilized for blood glucose monitoring.

Two novel and distinct classes of PBA-based polymers differing in the orientation of their PBA component relative to the polymer backbone were produced by a RAFT polymerization. The aqueous solubility of these polymers as well as their ability to suspend SWNT with surface coverage ranging up to 81% relative to NMP. The binding activity of the free PBA polymers is confirmed by ARS binding studies. Furthermore, after SWNT suspension, the formed sensors demonstrate a glucose response which occurs quickly and sensitively. The effect of differences in polymer structure, including polymer molecular weight and PBA position relative to the polymer backbone, on the resulting saccharide selectivity are demonstrated. Interestingly, the saccharide responses of the SWNT based nanosensors do not follow what is predicted for free boronic acids. Indeed, each SWNT-polymer system demonstrates distinct selectivity patterns to a library of saccharides, with one such system exhibiting enhanced selectivity towards glucose. The synthesis of this robust class of polymers is both simple and allows for precise structural control over the resulting species. Furthermore, this structural control allows for the formation of SWNT based nanosensors which demonstrate a tunable saccharide response. As such, this class of nanosensors represents the first example of a PBA-based polymer interacting with a nanoparticle to tailor the resulting saccharide response and ultimately to produce a sensitive, fast and continuous saccharide sensor with enhanced selectivity towards glucose.

### RAFT Polymerization of PBA Monomers

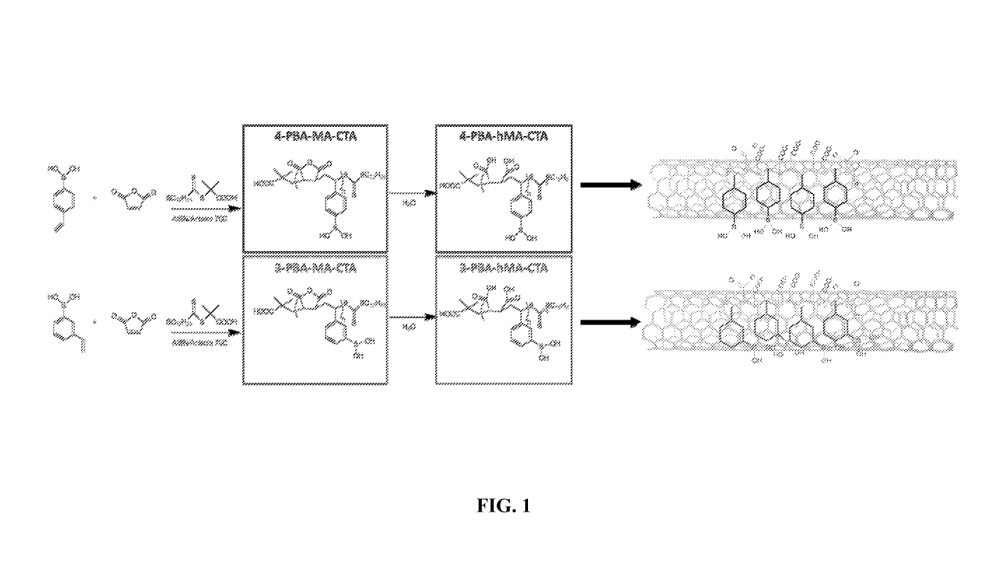

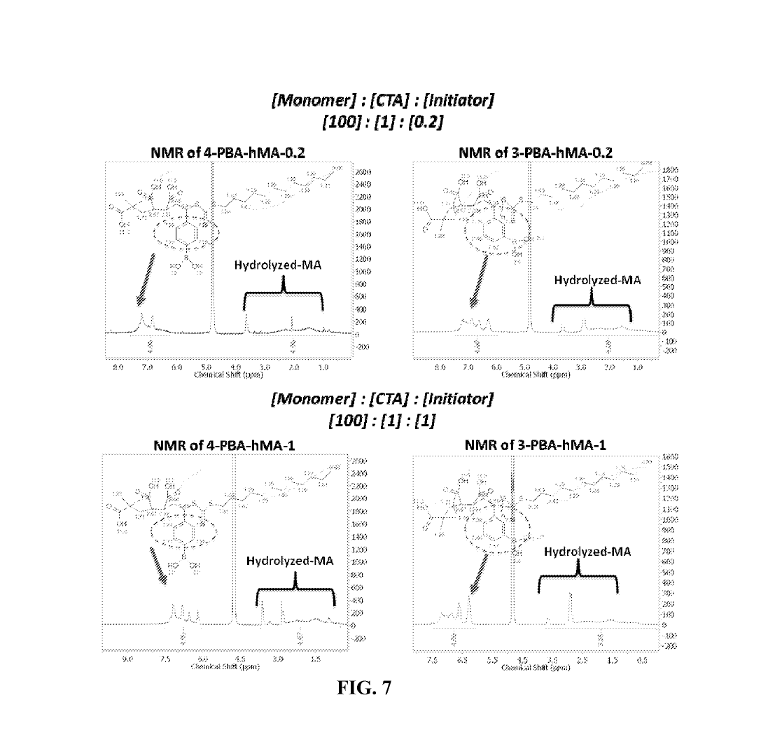

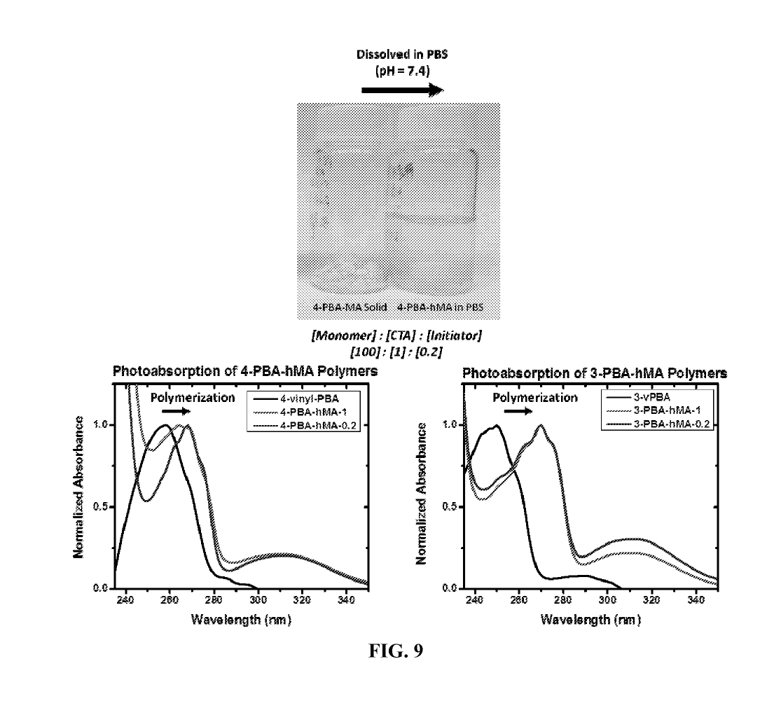

The RAFT polymerization reaction was conducted using two different initiator concentrations, 1 mole percent and 0.2 mole percent relative to the total monomer concentration in order to produce polymers of varying size. Generally, it polymer molecular weights follow an inverse dependence with initiator concentration. Thus, by using a smaller initiator concentration, the polymer grows to larger molecular weights. Each reaction was conducted under the same conditions, otherwise (FIG. 1). FIG. 1 shows that RAFT polymerization of vinyl-phenylboronic acid and maleic anhydride monomers was conducted followed by hydrolysis in order to produce a class of water soluble phenylboronic acid based polymers. These polymers were then directly used to impart water solubility to SWNT. Nuclear Magnetic Resonance (NMR) and Fourier Transform Infrared Spectroscopy (FTIR) demonstrate the formation of polymer having nearly 1:1 content of PBA and maleic anhydride. (FIGS. 7 and 8). FIG. 7 shows that NMR analysis confirms polymer formation in each case yielding approximately a 1:1 ratio of monomers. FIG. 8 shows that Fourier Transform Infrared Spectroscopy was used to characterize films made from hydrolyzed polymer solutions of each polymer system. Broad peaks ˜3550-3200 cm⁻¹ correlate to O—H stretches of the hydrolyzed backbone of the polymer and on the boronic acid. Aromatic C—H stretches from the boronic acid (˜3030 cm⁻¹) are also evident as are carboxylic C=0 stretches (1780-1710 cm⁻¹) from the hydrolyzed polymer backbone. After synthesis, the polymers are hydrolyzed in nanopure water (NP H₂O) or phosphate buffered saline (PBS) at 1 wt %. Interestingly, in all reactions, the photoabsorption spectra of the resulting polymer solutions demonstrate significant red-shifting of the PBA absorption peak relative to the monomer. (Table 1 and FIG. 9). FIG. 9 shows that simple stirring in either nanopure water (18 MS2) or PBS buffer (pH=7.4) hydrolyzes the formed polymer. Photoabsorption analysis of polymer solutions reveals that polymerization of the PBA monomer units induces a red-shifting of the associated photoabsorption peak. It should be noted that the photoabsorption spectra are normalized to the PBA peak in each case for concentration and to illustrate the peak position. This suggests that the polymerization confines the PBA such that significant electron-conduction may occur between the PBA components in polymeric form, as this red-shifting is also seen in other conductive conjugated polymers. See, Watanabe, A., et al., ELECTROCHROMISM OF POLYANILINE FILM PREPARED BY ELECTROCHEMICAL POLYMERIZATION. Macromolecules, 1987. 20(8): p. 1793-1796, which is incorporated by reference in its entirety. The solution is then analyzed using static and dynamic light scattering, yielding data about molecular weight and hydrodynamic radius, respectively, of the polymer in solution. The molecular weight ranges in the case of 3-vinylphenylboronic acid (3vPBA) and 4-vinylphenylboronic acid (4vPBA) are distinct, demonstrating a higher molecular weight of resulting polymer when 3vPBA monomer is used. (Table 1) This indicates a higher reaction efficiency in this case, as more monomer converted into polymeric form. As expected, using a smaller initiator concentration results in a larger molecular weight polymer in each case; this trend is confirmed by hydrodynamic radius measurements since the polymeric structures are similar to one another (Table 1). Zeta potential measurements demonstrate a negative zeta potential of approximately −38 mV in each case. This large negative zeta potential results from the significant content of deprotonated carboxylic acids in the polymer backbone, a fact that helps to stabilize the polymers in aqueous solution.

TABLE 1

| | | | | | | Relative |
|---|---|---|---|---|---|---|
| | | | | Hydrodynamic | ζ | Absorption |
| Polymer | [Monomer] | [CTA] | [Initiator] | Radius (nm) | potential (mV) | Shift (nm) |
| 4-PBA-hMA-0.2 | 100 | 1 | 0.2 | 220 ± 4.9 | −38.52 ± 0.15 | 10 |
| 4-PBA-hMA-1 | 100 | 1 | 1 | 87.5 ± 22.5 | −38.27 ± 0.21 | 6 |
| 3-PBA-hMA-0.2 | 100 | 1 | 0.2 | 398.5 ± 19.6 | −38.43 ± 0.06 | 20 |
| 3-PBA-hMA-1 | 100 | 1 | 1 | 309.08 ± 18.2 | −38.38 ± 0.1 | 20 |

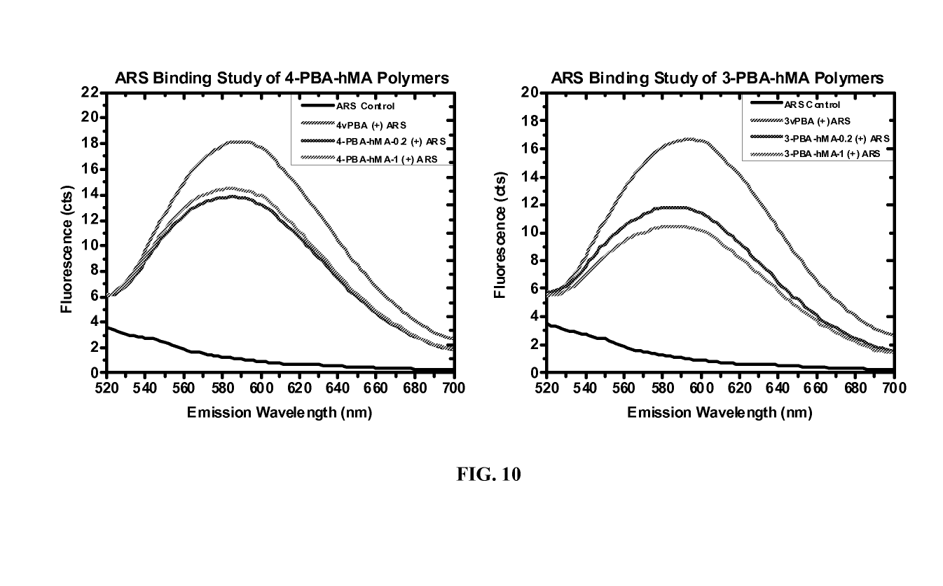

ARS binding studies were used to confirm that the binding ability of the boronic acid components was not affect by the polymerization process (FIG. 10). FIG. 10 shows that ARS binding studies illustrate the conserved ability of the PBA monomer to form diol bonds. This is an important fact when considering saccharide binding and helps to show that RAFT polymerization does not inhibit the activity of the PBA diols. Significantly, all polymers induce a strong ARS fluorescence when the two components are mixed. This indicates that the PBA successfully undergo diol-bond formation with the ARS and, therefore, should effectively bind to saccharides. This is an important point that helps to illustrate the simplicity and robustness of this synthetic method.

### Formulation of Water Soluble Polymer Nanosensor

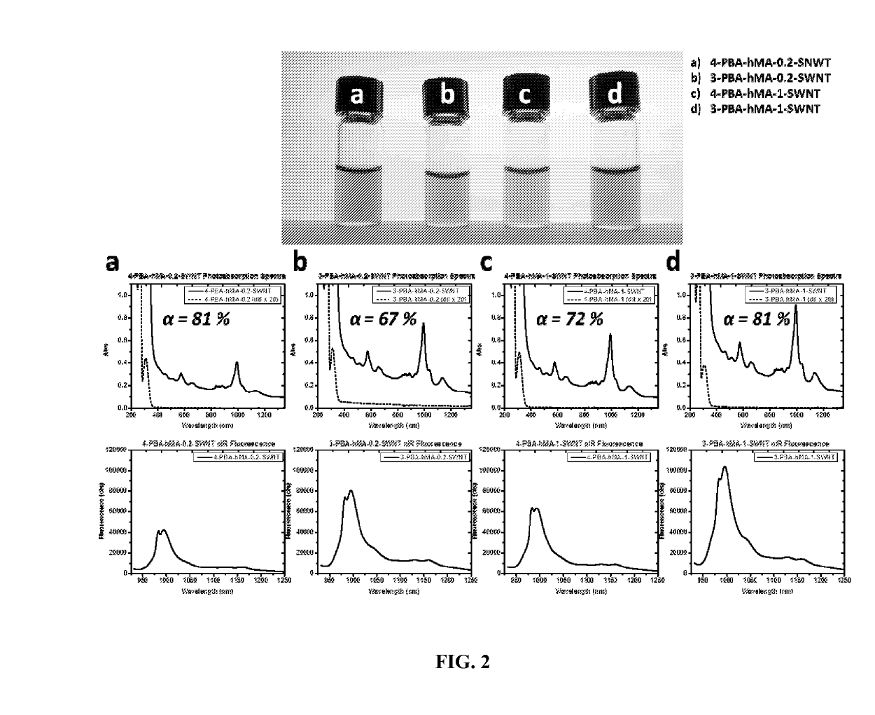

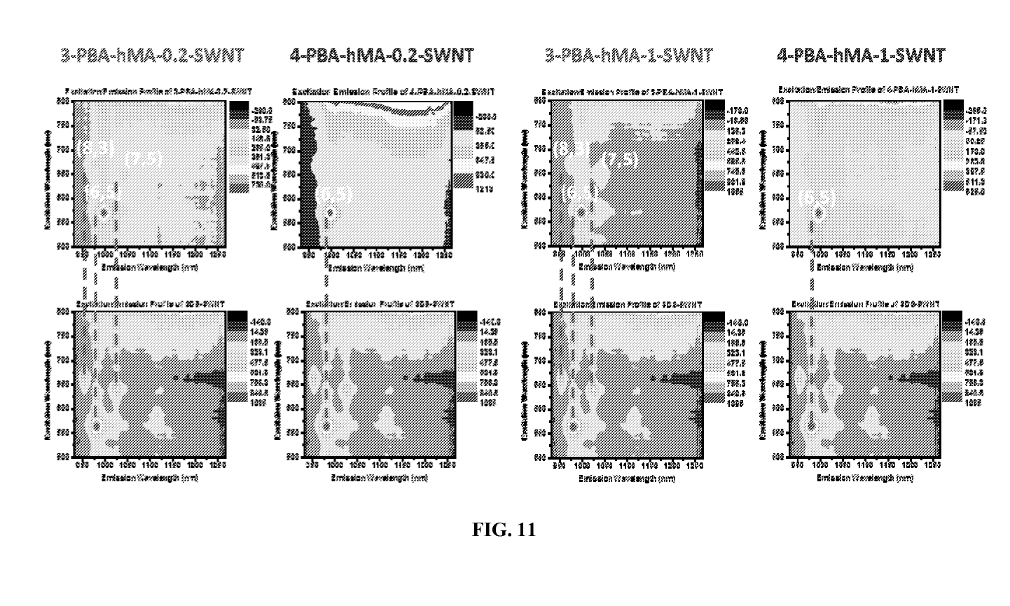

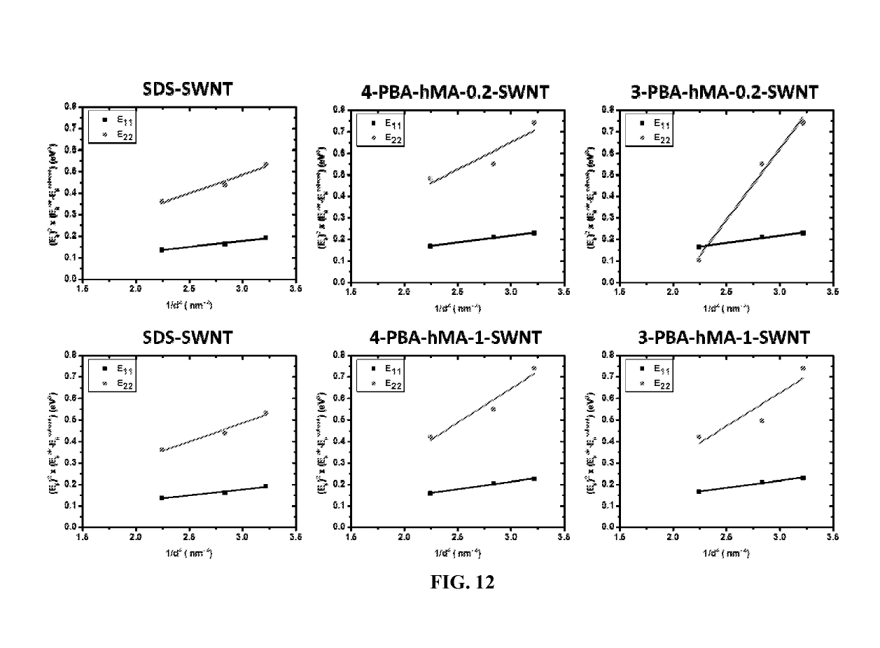

Utilizing the strong π-π stacking interaction of the PBA and the SWNT, these polymers were demonstrated to successfully suspend SWNT by direct sonication, as indicated by the photoabsorption and nIR fluorescent spectra presented in FIG. 2. FIG. 2 shows that all polymers give stable aqueous suspensions of polymer SWNT, as can be observed by the photoabsorption and nIR fluorescent spectra observed from each polymer-SWNT suspension. Interestingly, it appears that polymers made using 3-vinylphenylboronic acid suspend SWNT in higher concentrations than their 4-vinylboronic acid, despite having similar chemical structures as observed by NMR analysis. nIR fluorescent excitation/emission maps were also taken for each polymer-SWNT system. Previous work has demonstrated that this analysis can be used to estimate the SWNT surface coverage demonstrated by each polymer relative to NMP. Because of its high surface packing on SWNT, NMP is taken as a standard for comparison and set at 100% SWNT surface coverage. From this, the surface coverage relative to NMP (a) can be determined (FIGS. 11 and 12, summarized as inset of photoabsorption spectra of FIG. 2). See, Choi, J. H. and M. S. Strano, Solvatochromism in single-walled carbon nanotubes. Applied Physics Letters, 2007. 90(22), and Hilmer, A. J., et al., Charge Transfer Structure-Reactivity Dependence of Fullerene-Single-Walled Carbon Nanotube Heterojunctions. Journal of the American Chemical Society, 2013. 135(32): p. 11901-11910, each of which is incorporated by reference in its entirety. FIG. 11 shows that fluorescent excitation/emission mapping demonstrates the successful SWNT suspension formation. This analysis can also be utilized in previous publications. The plot for SDS-SWNT is also presented for comparison. FIG. 12 shows that plotting E₁₁ v 1/d⁴ allows for the assignment of relative SWNT surface coverage assuming 100% surface coverage by NMP. The plot for SDS-SWNT is also presented for comparison.

The concept behind designing this class of polymers was to create systems where the primary interaction site between the polymer and the SWNT also functioned as the saccharide receptor. Previous studies relied on polymers to tether receptors to the SWNT surface in order to induce detection. See, Yoon, H., et al., Periplasmic Binding Proteins as Optical Modulators of Single-Walled Carbon Nanotube Fluorescence: Amplifying a Nanoscale Actuator. Angewandte Chemie-International Edition. 50(8): p. 1828-1831, which is incorporated by reference in its entirety. In this type of nanosensor, the interaction of the analyte receptor and the SWNT fluorophore are governed by the polymer tether. However, the interaction of the PBA and the SWNT previously discovered suggests significant adsorption of the PBA on the side-wall of the SWNT even through surfactant corona. As a result, this system is primly suited to designing a polymer system where the receptor also serves as the SWNT docking site. Utilizing this design, it was thought that sensor sensitivity could be significantly enhanced.

### Saccharide Detection

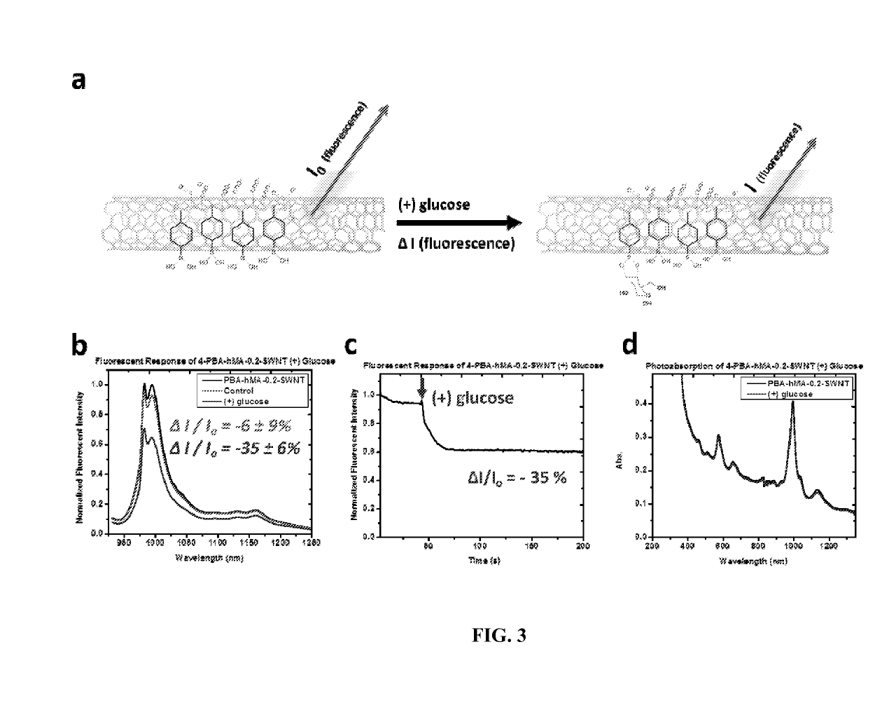

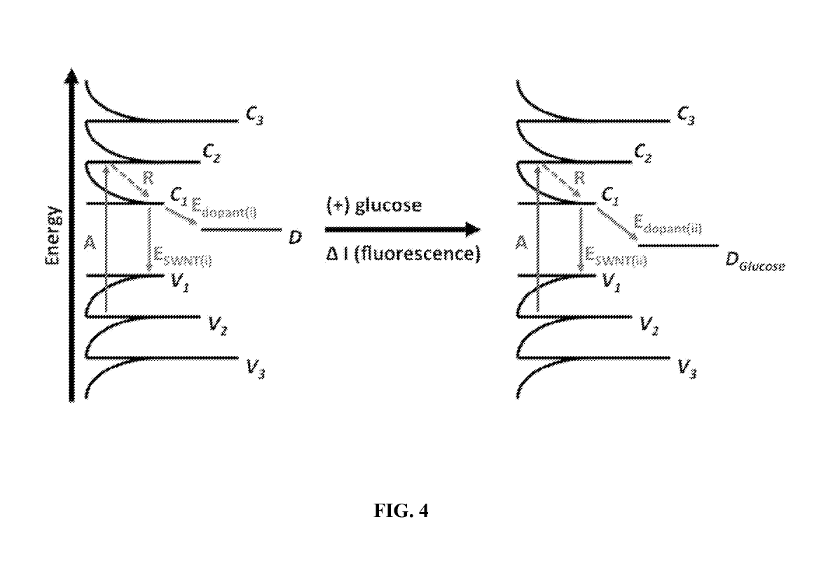

FIG. 3 demonstrates that this system allows for a glucose induced nIR fluorescent quenching of 35±6% relative to 50 mM glucose addition. FIG. 3A shows a schematic of a 4-vinyl phenylboronic acid polymer derivative-SWNT complex illustrating a proposed mechanism for glucose binding to the boronic acid component of the nanosensor. Here, the binding changes the local dielectric constant of the nanosensor and ultimately induces a fluorescent quenching of the SWNT fluorophore (FIG. 3B). This fluorescent quenching occurs rapidly after the addition of glucose, as is illustrated in the transient fluorescent data in c. Furthermore, figure d shows that the mechanism of fluorescent quenching is not due to SWNT aggregation or destabilization, as no observable change in the photoabsorption spectra is observed after glucose addition. Furthermore, transient measurements indicate that this induced quenching occurs very quickly, reaching steady state quenched fluorescence in less than 25 second. Photoabsorption analysis before and after glucose addition indicates no significant change in the peak intensity or position of the SWNT absorption peaks. This suggests that the observed fluorescent quenching does not result from SWNT aggregation. Therefore, it can be asserted that binding of the glucose to the PBA induces a change in the reduction potential of the PBA dopant state (FIG. 4). FIG. 4 shows that photoabsorption (A) induces an electronic excitement of the SWNT. Internal relaxation (R) can then occur followed either by fluorescence (ESWNT(i)) or energy transfer between the excited SWNT and the dopant (D) phenylboronic acid polymer electronic state. After glucose binding, the reduction potential of the dopant state of the polymer is reduced, causing an increased in energy transfer to the polymer. Hence, the energy transfer to the dopant (Edopant) increases and energy emission through SWNT fluorescence (ESWNT) decreases. The result is a fluorescent quenching. Hence, Edopant(i)<Edopant(ii) and ESWNT(i)>ESWNT(ii). This modulation of the reduction potential induces an increased probability of excited state electron transfer between the SWNT and the PBA. Ultimately, this causes a decrease in the radiative relaxation (or fluorescence) of excited electrons in the SWNT, or a fluorescent quenching.

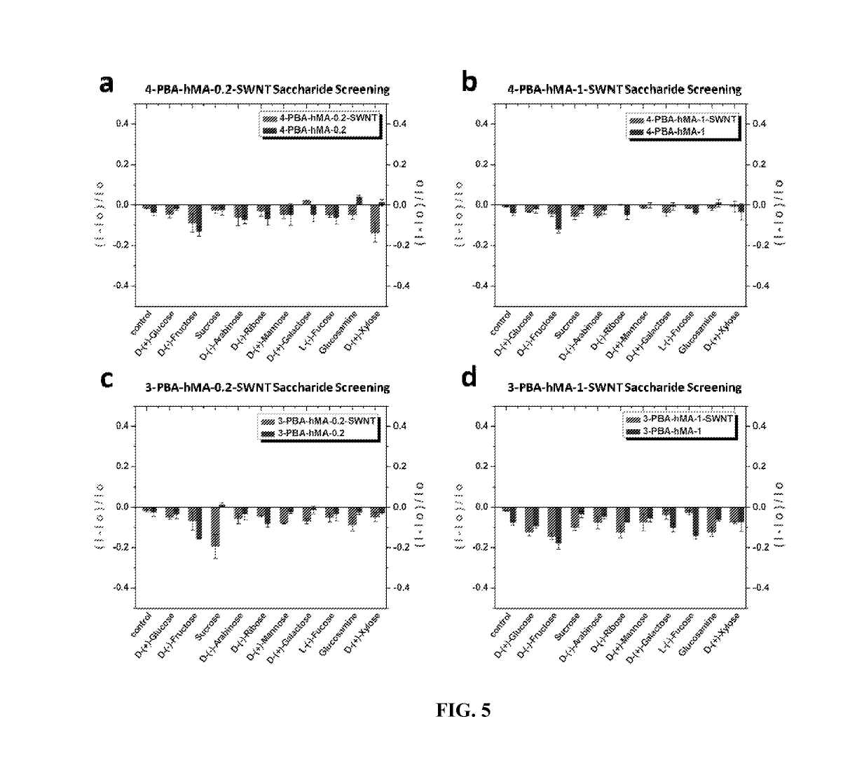

Interestingly, the saccharide selectivity profiles of polymer-SWNT sensors do not follow the predicted tend of free PBA. This is illustrated in FIG. 5, where plots of induced fluorescent response of polymer-SWNT nanosensors is plotted with competitive saccharide-ARS binding results for free polymers. FIG. 5 shows that the saccharide binding profiles of all polymers-SWNT are distinct both from one another and from the free polymers (probed using competitive binding of each saccharide with ARS bound polymers). FIG. 5A shows that the saccharide binding profile of 4-vPBA-hMA-0.2-SWNT demonstrates a bias towards D-(+)-xylose followed by D-(−)-fructose. FIG. 5B shows that simply decreasing the initiator concentration (4-vPBA-hMA-1-SWNT), and therefore polymer size, changes the response such that the polymer does not significantly respond to any saccharide in this library. FIG. 5C shows that changing the position of the PBA, relative to the polymer backbone significantly changes the observed saccharide response. Here, 3-PBA-hMA-0.2-SWNT shows the strongest response to sucrose. FIG. 5D shows that again, conserving the PBA position relative to the polymer backbone but changing the initiator concentration modifies the saccharide binding profile to favor D-(−)-fructose and D-(+)-glucose the strongest. Significantly, the binding profiles of all free polymers favors D-(−)-fructose binding in all cases. This implies the association with the SWNT modifies the relative binding constants of all saccharides with these PBA polymers.

Firstly, it is obvious that each polymer demonstrates a unique saccharide binding profile. However, none of the polymer-SWNT sensors demonstrate a saccharide binding profile which matches that of the free polymer-saccharide binding profiles. Specifically, as expected for the free polymer, the largest fluorescent modulation of the ARS bound polymer occurs when fructose is added. This results from the fructose having the strongest binding interaction with free boronic acids, allowing fructose to displace the most ARS from the formed diol bond with the PBA-polymers. However, when the polymer associates with the SWNT, this selectivity profile changes in each case.

For the cases of polymer formed using lower concentrations of initiator, and therefore higher molecular weight polymers, the selectivity profile depends distinctly on orienting the PBA relative to the polymer backbone. In the case of 4-PBA-hMA-0.2-SWNT (nomenclature=4vinyl-PBA-hydrolyzed maleic anhydride-[initiator]-SWNT suspension), the largest response of the nanosensor comes from D-(+)-xylose. Significantly, this strong and preferential response to D-(+)-xylose is not observed when the ARS-bound free polymer is profiled. This indicates that adsorption of the polymer to the SWNT surface changes the selectivity of the resulting nanosensor. Analyzing the response profile of 3-PBA-hMA-0.2-SWNT, it is evident that changing the orientation of the PBA relative to the polymer backbone alters the selectivity of the resulting nanosensor such that it responds most preferentially to sucrose rather than D-(+)-xylose. As such, it appears that changing the PBA orientation relative to the polymer backbone, and therefore the saccharide binding orientation relative to the polymer backbone, affects each saccharide-polymer-SWNT binding constant distinctly.

Similarly, when a larger concentration of initiator is used, and therefore a smaller polymer is formed, the saccharide binding profiles of the resulting nanosensors changes from what is observed using the larger molecular weight counterparts. Specifically, the saccharide binding profile of 4-PBA-hMA-1-SWNT shows that this system responds weakly to all the saccharides in the testing library. However, by again changing the orientation of the PBA relative to the polymer backbone, it is possible to alter the saccharide selectivity such that the resulting nanosensor response most strongly to D-(+)-glucose, D-(−)-fructose and D-(+)-glucosamine. Again, this selectivity does not follow the selectivity predicted from the binding energies of free PBA. Rather, the combination of the polymerization and association with the SWNT alters the binding energy to give unique selectivity profiles for each nanosensor system. A number of factors may contribute to these differences in saccharide binding between polymers, as well as their deviation from what is expected for free PBA. One such factor is the orientation of the boronic acid relative to the SWNT axis. This would likely effect the steric hindrance that saccharides experience when solvating into the SWNT corona to bind with the PBA component of the polymer. Ultimately, this induced hindrance would affect saccharides differently depending on the position of the saccharide diols relative to the orientation of the boronic acid. With this in mind, it is likely that shorter chain polymers would be packed differently on the SWNT from their larger chain counterparts. This packing would also effect the orientation of the PBA relative to the SWNT axis and therefore the steric hindrance in a saccharide specific manner. Another key component of this system is the interaction of the molecular orbitals of the SWNT and the phenyl-ring of the PBA. This interaction allows for the translation of saccharide binding with the PBA into an observable modulation of the SWNT fluorescence. As such, it should be possible to tune the saccharide response to be highly selective by controlling the molecular weight and orientation of the PBA relative to the polymer backbone.

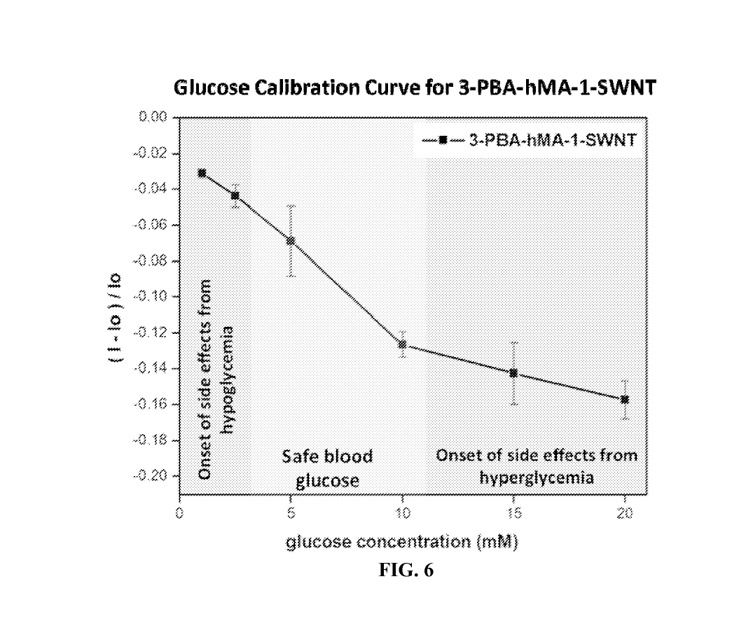

This glucose binding response was further probed to analyze its sensitivity. As is demonstrated in FIG. 6, 3-PBA-hMA-1-SWNT demonstrates sensitivity to glucose concentrations down to and including 2.5 mM glucose. This surpasses the lower limit of what had previously been demonstrated using similar nanosensor. See, Yoon, H., et al., Periplasmic Binding Proteins as Optical Modulators of Single-Walled Carbon Nanotube Fluorescence: Amplifying a Nanoscale Actuator. Angewandte Chemie-International Edition. 50(8): p. 1828-1831, which is incorporated by reference in its entirety. Significantly, this indicates that this sensor is highly promising for monitoring fluctuations of glucose levels spanning the hypoglycemic and hyperglycemic ranges. Having sensitivity to both lower and upper limits of physiological glucose concentrations is paramount to creating closed loop continuous blood glucose monitoring systems for patients suffering from diabetes.

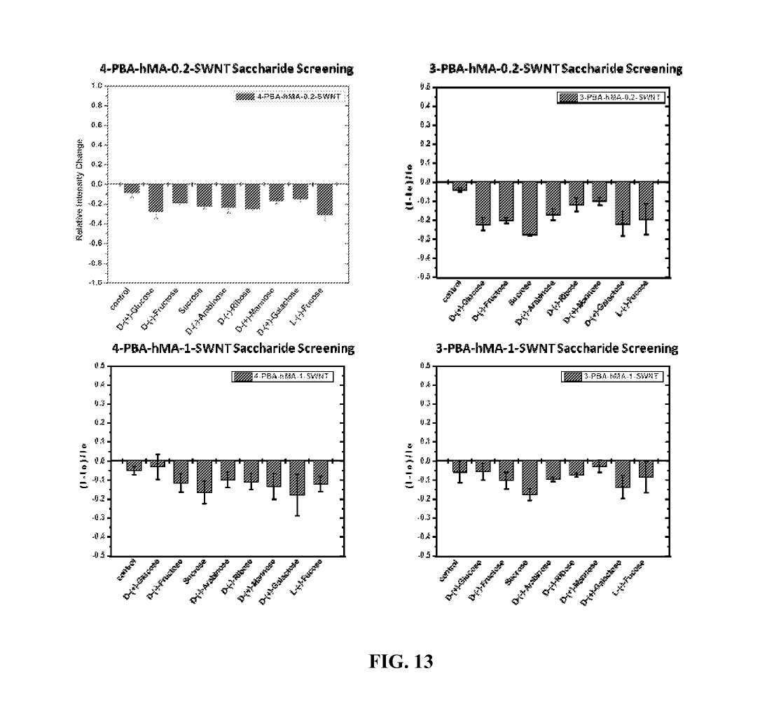

In FIG. 13, saccharide screening done at pH=1 demonstrates that significantly changing the pH alters the binding profile of each polymer-SWNT system. This is another important factor which should be considered when tuning the selectivity of this class of nanosensor.

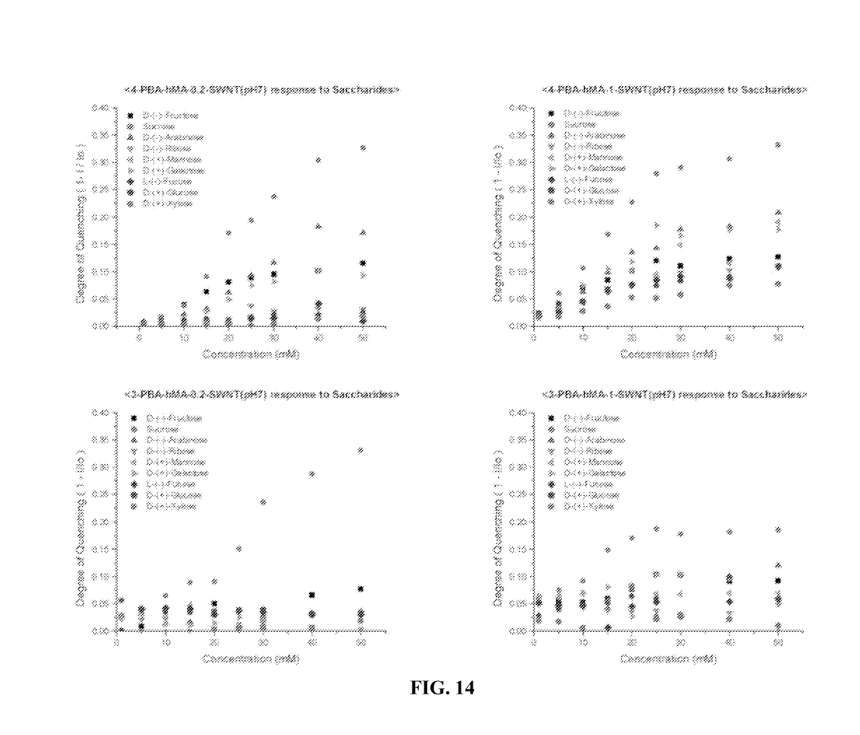

FIG. 14 shows the calibration curves for saccharides. By varying the polymer length and the location of boronic acid, it was possible to achieve a high selectivity toward a certain saccharide. The RAFT polymerization reaction was conducted using two different initiator concentrations, 1 mole percent and 0.2 mole percent relative to the total monomer concentration in order to produce polymers of varying size.

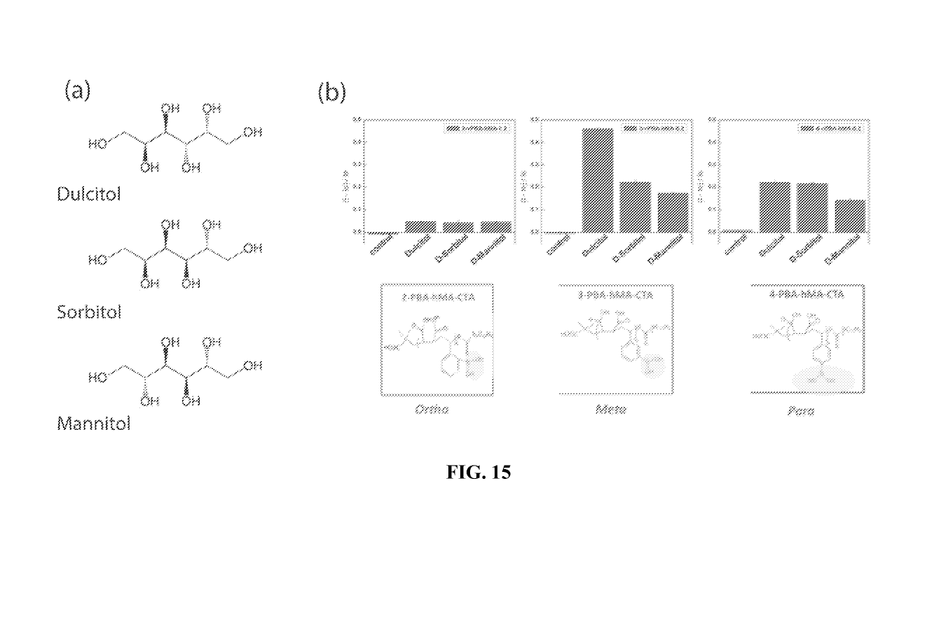

FIG. 15 shows the relation between the response to sugar alcohol and the location of the boronic acid. Three sugar alcohols tested in this study have very similar structures (FIG. 15A). Three polymers were synthesized with similar molecular weights by RAFT polymerization. Only difference among these polymers is the location of the boronic acid, which is shown to have an enormous impact on the responses. When boronic acid is located at the meta position, the polymer-SWNT complex can detect the subtle differences among these sugar alcohols (FIG. 15B).

In conclusion, a simple and robust RAFT polymerization process can produce two novel and distinct classes of PBA-based polymers and allows the polymers to form stable aqueous suspensions of PBA polymer-SWNT nanosensors. Interestingly, the saccharide binding selectivity of the resulting nanosensors did not follow the expected trend for free boronic acids. The polymer molecular weight and the orientation of the PBA relative to the polymer backbone are two components which can be manipulated in order to tune the saccharide selectivity of the resulting polymer-SWNT nanosensors. Manipulating these parameters yielded a nanosensor which demonstrated enhanced selectivity towards D-(+)-glucose. This sensor was shown to be sensitive as well as stable during continuous probing. As such, this class of polymers holds a great deal of promise for effectively forming a durable and sensitive nanosensor with tunable selectivity to various saccharides. Furthermore, one such sensor demonstrates enhanced selectivity towards D-(+)-glucose and a suppressed selectivity towards D-(−)-fructose compared to free boronic acids, pointing the way towards a reliable continuous D-(+)-glucose sensing nanosensor. Ultimately, it is hoped that this system can function in vivo in order to provide continuous and real-time blood glucose levels to improve the quality of life for and help eliminate the many potentially fatal side-effect of patients with diabetes.

### TECHNICAL DETAILS

I) RAFT Polymerization of PBA Monomers:

Maleic anhydride (5 mmol) was combined with the desired vinylphenylboronic acid derivative (5 mmol) to achieve a total monomer amount of 10 mmol. The mixture was then dissolved in 10 mls of anisole. The desired relative amount of initiator 2,2′-Azobis(2-methylpropionitrile) (AIBN, 0.02 mmol and 1 mmol used in this study) and 2-(Dodecylthiocarbonothioylthio)-2-methylpropionic acid (chain transfer agent (CTA), 0.1 mmol) was placed in a 25 ml roundbottom flask equipped with a stirbar and then mixed with the dissolved maleic anhydride and phenylboronic acid monomers. The mixture was allowed to dissolve prior to removing air from the reaction vessel using a roughing pump (˜45 min) followed by sparging with UHP N₂ for 30 min under vigorous stirring. Thermal induced radical polymerization was then conducted by placing the de-gassed stirring mixture into a 70 C oil bath. RAFT was conducted for several hours until polymer sedimentation. The vessel was opened to air in order to stop the reaction and allowed to cool to room temperature. The solid was then dissolved in anisole followed by recrystallization in a 20 fold volumetric excess of cold diethyl ether. After recrystallization, the product was dried overnight in a vacuum dessicator prior to characterization. All chemicals were purchased from Sigma Aldrich.

Spectroscopic Characterization:

H¹ spectra was assigned using a VARIAN Inova (500 mHz) NMR. All NMR were conducted in 1 M NaOD (Sigma). FTIR was conducted using attenuated total reflection infrared spectroscopy (ATR-IR) with a Thermo Nicolet 4700 spectrometer. Polymers were dissolved at 1 wt % in nanopure water (NP H₂O) followed by drop-drying solution onto a glass microscope slide for polymer film analysis. UV-VIS-nIR photoabsorption spectroscopy was conducted using a Shimadzu UV-3101PC spectrometer and using 1 cm pathlength quartz cuvettes (Starna). nIR fluorescent measurements were taken using an inverted Zeiss AxioVision microscope coupled to a Princeton Instruments InGaAs OMA V array detector through a PI-Action SP2500 spectrometer. Visible fluorescence measurements of alazirin red (ARS) was accomplished using a Varioskan Plate Reader scanning from 520-700 nm while exciting at 495 nm for is.

Light Scattering:

Zeta potential and light scattering data were performed on 1 wt % polymer solutions. Zeta potential measurements were accomplished using a Zeta PALS from Brookhaven Instrument Corporation. Dynamic light scattering was performed using the same instrumentation as was used for Zeta potential analysis, and was performed in order to analyze the hydrodynamic radius. Static light scattering was performed using a Brookhaven Instrument Corporation model BI-200SM using a 636.8 nm diode laser and was performed in order to determine polymer molecular weight.

II) Formulation of Water Soluble Polymer Nanosensor:

Polymers were dissolved at 1 wt % in NP H₂O and combined with 1 mg SWNT (Southwest Nano SG65) per milliliter of polymer solution. The mixture was then probe tip sonicated (6 mm tip, Cole Parmer) at 0.8 W/ml for 30 min in an ice-bath. After 30 min sonication, the ice-bath was refilled with ice and the solution was sonicated for an additional 30 min. Following sonication, the dispersed polymer-SWNT solution was ultracentrifuged at 187,000×g for 4 hrs. The top 80% of volume of ultracentrifuged material was then isolated. After isolation, the pH was tuned to 7.4 by dialyzing against PBA buffer.

Other embodiments are within the scope of the following claims.

1. A composition for sensing an analyte, comprising:
a photoluminescent nanostructure complexed to a sensing polymer, wherein the sensing polymer is a copolymer including monomer units having a boronic acid moiety and non-covalently bound to the photoluminescent nanostructure;
wherein the composition is capable of selectively binding the analyte, and the composition undergoes a substantial conformational change when binding the analyte.

2. The composition of claim 1, wherein the photoluminescent nanostructure is a carbon nanotube.

3. The composition of claim 2, wherein the carbon nanotube is a SWNT.

4. The composition of claim 3, wherein the boronic acid moiety is a phenylboronic acid.

5. The composition of claim 4, wherein the analyte is a saccharide.

6. The composition of claim 5, wherein the saccharide is glucose.

7. A method of synthesizing a composition for sensing an analyte, comprising:
selecting a concentration a initiator and a boronic acid derivative,
conducting polymerization of a monomer and the boronic acid derivative, wherein the resulting polymer has a selectivity to an analyte,
and mixing with a photoluminescent nanostructure.

8. The method of claim 7, wherein the photoluminescent nanostructure is a carbon nanotube.

9. The method of claim 8, wherein the carbon nanotube is a SWNT.

10. The method of claim 9, wherein the boronic acid is a phenylboronic acid.

11. The method of claim 10, wherein the analyte is a saccharide.

12. The method of claim 11, wherein the saccharide is glucose.

13. A method for sensing an analyte, comprising:
providing a composition, wherein the composition includes:
a photoluminescent nanostructure complexed to a sensing polymer, wherein the sensing polymer is a copolymer including monomer units having a boronic acid moiety and non-covalently bound to the photoluminescent nanostructure;
wherein the composition is capable of selectively binding the analyte, and the composition undergoes a substantial conformational change when binding the analyte; and
contacting the composition with a sample suspected of containing the analyte.

14. The method of claim 13, wherein the photoluminescent nanostructure is a carbon nanotube.

15. The method of claim 14, wherein the carbon nanotube is a SWNT.

16. The method of claim 15, wherein the boronic acid moiety is a phenylboronic acid.

17. The method of claim 16, wherein the analyte is a saccharide.

18. The method of claim 17, wherein the saccharide is glucose.

19. A composition for sensing an analyte, comprising a complex, wherein the complex includes a photoluminescent nanostructure in an aqueous dispersion and a boronic acid capable of selectively reacting with an analyte.

20.-24. (canceled)

25. A device for sensing an analyte, comprising:
a hydrogel particle encapsulating a composition, wherein the composition includes a complex, wherein the complex includes a photoluminescent nanostructure in an aqueous dispersion and a boronic acid capable of selectively reacting with an analyte.

26.-30. (canceled)

31. A method for sensing an analyte, comprising:
providing a composition of claim 1; and
contacting the composition with a sample suspected of containing the analyte.

32.-50. (canceled)
